(19) United States
(12) Patent Application Publication
(10) Pub. No.: US 2006/0024312 A1
(43) Pub. Date: Feb. 2, 2006

(54) Methods for enhancing learning and memory

(75) Inventors: Cristina Alberini, New York, NY (US); Ana Garcia-Osta, New York, NY (US); Jeffrey Kleim, Lethbridge (CA)

Correspondence Address: DARBY & DARBY P.C., P. O. BOX 5257, NEW YORK, NY, 10150-5257, US

(73) Assignee: Mount Sinai School of Medicine of New York University, New York, NY (US)

(21) Appl. No.: 11/172,410

(22) Filed: Jun. 29, 2005

Related U.S. Application Data
(63) Continuation-in-part of application No. PCT/US04/05006, filed on Feb. 20, 2004.

(51) Int. Cl. A61K 39/395; C12Q 1/68; C07H 21/04; C12P 21/06; C12N 9/12; C12N 5/08

(52) U.S. Cl. 424/146.1; 435/69.1; 435/194; 435/368; 435/320.1; 536/23.2; 435/6

(57) ABSTRACT

Methods of maintaining or enhancing memory or learning in a mammal by activating the receptor tyrosine kinase muscle-specific kinase (MuSK) in the brain are disclosed. Also disclosed are methods of treating a disease or condition associated with memory loss or a neurological impairment by administering an effective amount of a MuSK-activating agent to a subject, such as a human, in need of such treatment. The invention also pertains to methods of identifying compounds that maintain or enhance memory or learning or that enhance the recovery from a neurological impairment such as stroke. MuSK, an agrin receptor known to be important in neuromuscular junction formation and function, was isolated from the brain and determined to play an essential role to memory consolidation and learning.

This application is a continuation-in-part application of International application no. PCT/US2004/005006, filed Feb. 20, 2004, which is hereby incorporated by reference.

The research leading to this invention was supported, in part, by Grant No. R01 MH65635 awarded by the National Institute of Mental Health. Accordingly, the United States government may have certain rights to this invention.

### FIELD OF THE INVENTION

The present invention pertains to the identification of muscle-specific kinase (MuSK) expression in the brain and, in particular, the increase of its expression with memory and learning. The invention also pertains to methods of maintaining or enhancing memory or learning in a mammal comprising activating MuSK in the brain of the mammal. The invention also pertains to methods of treating stroke comprising administering an effective amount of a MuSK-activating agent to a subject in need of such treatment.

### BACKGROUND OF THE INVENTION

A function of the adult brain known to involve synaptic changes and perhaps synaptogenesis is memory formation. In invertebrates, as in mammals, the formation of new memories requires a process known as consolidation, whereby incoming information is transformed into stable modifications. This consolidation process requires a cascade of gene expression and is accompanied by long-term changes in synaptic strength, which is likely based on changes in synaptic structures and/or numbers. These same neural processes are also thought to underlie restoration of function after brain damage, such as recovery from stroke.

In the adult mammalian brain, the hippocampus is known to mediate the consolidation of contextual and spatial memories. Identifying mediators of memory consolidation is essential for developing treatments for diseases or conditions associated with memory loss such as Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, Parkinson's disease, and stroke. Identification of such mediators is also essential for understanding synapse formation and for developing treatments for conditions associated with neurological impairment such as spinal cord injury, brain trauma, ischemia, shock, head trauma, neuronal injury, neuronal toxicity, neuronal degeneration and stroke.

To identify genes that are regulated during memory consolidation, DNA microarray hybridizations were screened for genes differentially expressed in the hippocampus after inhibitory avoidance learning. The present invention describes the identification of muscle-specific kinase (MuSK) as a gene that is upregulated during inhibitory avoidance learning; the present invention employs this discovery in methods of treating memory and neurological impairments.

Muscle-Specific Kinase (MuSK)

MuSK is a receptor tyrosine kinase that was first cloned in rat, human (Valenzuela et al., (1995) Neuron 15: 573-584) and mouse (Ganju et al., (1995) Oncogene 11: 281-290) and more recently in Xenopus (Fu et al., (1999) Eur. J. Neurosci. 11: 373-382) and chicken (Ip et al., (2000) Mol. Cell Neurosci. 16: 661-673). Because of its high levels of expression in early muscle development, in muscle fibers after denervation and in neuromuscular junctions (NMJ), compared to a variety of other adult tissues (Valenzuela et al., (1995) Neuron 15: 573:584), it was concluded that MuSK was selectively expressed in muscle and not in the mammalian brain (Smith and Hilgenberg, (2002) NeuroReport 13(12): 1485-95). However, in chicken and Xenopus MuSK mRNA has also been found in other tissues, including adult spleen and lung, and appears to be highly expressed during development in neural tissues (Fu et al., (1999) Eur. J. Neurosci. 11: 373-382; Ip et al., (2000) Mol. Cell Neurosci. 16: 661-673) and in liver (Ip et al., (2000) Mol. Cell Neurosci. 16: 661-673).

Numerous studies have contributed to uncovering the function of MuSK in muscle cells and particularly at NMJ, where it has been found to play an essential role in directing the accumulation of acetylcholinesterase receptors (AChRs) in the post-synaptic apparatus (DeChiara et al., (1996) Cell 85: 501-512; Burden, (2002) J. Neurobiol. 53: 501-511; Sanes and Luchtman, (1999) Annu. Rev. Neurosci. 22: 389-442). Thus it emerged that the formation of NMJs occurs through a series of steps, which initiates as soon as the tips of the growing motor neurons contact the muscle fiber. First, the motor nerve terminals release the heparan-sulfate proteoglycan agrin and the neuregulin ARIA (AChR inducing activity), both of which are required to mediate the post-synaptic specialization. Second, agrin interacts with MuSK and other proteins and induces MuSK phosphorylation, which mediates the clustering of membrane-expressed proteins, including AChRs. Third, ARIA increases the local transcription of AChRs at the subsynaptic region. Finally, MuSK activates intracellular pathways that mediate postsynaptic and presynaptic differentiation. Both agrin and MuSK are essential for NMJ formation, as knock-out mice of agrin or MuSK do not form neuromuscular synapses and die soon after birth.

Agrin is widely expressed in the nervous system (O'Connor et al., (1994) J. Neurosci 14: 1141-52) as well as in non-neuronal tissues (Hoch et al., (1993) Neuron 11 (3):479-490). Several works have suggested that in the nervous system agrin participates in the genesis of neuronal synapses. In the developing brain, agrin expression peaks during periods of synapse formation. In the mature brain, agrin levels are highest in structures, such as hippocampus and cortex, which are known to maintain a high degree of synaptic plasticity throughout life (Biroc et al., (1993) Brain Res Dev Brain Res 75(l):119-29; Stone and Nicolics, (1995) J Neurosci, 15; Smith and Hilgenberg, (2002) NeuroReport 13(12): 1485-95). Studies based on the suppression of agrin expression have led to unclear conclusions. On one hand, the knock-down of agrin in neuronal culture results in a decrease in the GABA_{A} receptors and clusters (Ferreira, (1999) J. Cell Sci. 112: 4729-38), indicating a role for agrin in GABAergic synapses. On the other hand, agrin knockout mice have a smaller although grossly normal brain, suggesting that in the brain, unlike at the NMJ, the function of agrin might be redundant.

Nevertheless, in the nervous system, the intemeuronal receptor for agrin has yet to be identified (Smith and Hilgenberg, (2002) NeuroReport 13(12): 1485-95).

The present invention describes the cloning of two isoforms of MuSK, a known agrin receptor, in the brain and describes MuSK expression in the brain and at neuronal synapses and presents data showing that brain MuSK plays an essential role in forming long-term memory.

The present invention also describes that MuSK may be acting, among possible other pathways, through the cAMP response element binding protein (CREB) pathway. CREB has been shown to play a critical role in memory formation (Alberini (1999) J. Exp. Biol. 202, 2887-2891; Silva et al. (1998) Ann. Rev. Neurosci. 21, 127-148; Kandel, (2001) Science 294, 1030-1038).

### SUMMARY OF THE INVENTION

The present invention concerns the discovery that MuSK is expressed in the brain and that MuSK expression in the brain increases with learning and memory.

The present invention provides methods of maintaining or enhancing memory or learning in a mammal comprising activating muscle-specific kinase (MuSK) in the brain of the mammal. In a specific embodiment of the invention the section of the brain in which MuSK is activated is the hippocampus. In other embodiments of the invention the type of memory enhanced is long-term memory, working memory or memory consolidation.

In one embodiment of the invention the method of maintaining or enhancing memory or learning in the mammal comprises increasing MuSK expression in the brain of the mammal. In one embodiment, such increase in MuSK expression is achieved by stabilizing or preventing the degradation of MuSK polypeptide or mRNA. In yet another embodiment, the method of maintaining or enhancing memory or learning in the mammal comprises administering a MuSK-activating agent to said mammal. In one embodiment of the invention, the MuSK-activating agent is a MuSK-activating antibody.

In a specific embodiment of the invention MuSK is a polypeptide comprising the amino acid sequence SEQ ID NO: 2, which is encoded by the nucleotide sequence SEQ ID NO: 20 (and SEQ ID NO: 1).

The present invention also provides methods of treating a disease or condition associated with memory loss comprising administering an effective amount of a MuSK-activating agent to a subject in need of such treatment. In preferred embodiments, such subjects are mammals or even more preferably humans. In one embodiment such methods are achieved by administering an effective amount of a MuSK-activating agent to a subject in need of such treatment. In yet another embodiment, the MuSK-activating agent is selected from the group consisting of a MuSK-activating antibody, an inhibitor of a MuSK inhibitor, and a MuSK-activating small molecule. In yet another embodiment of the invention, the disease or condition associated with memory loss is selected from the group consisting of Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, Parkinson's disease, and stroke. In one embodiment, the Alzheimer's disease is in its early stage. In yet a further embodiment, the neurological impairment is selected from the group consisting of spinal cord injury, brain trauma, ischemia, shock, head trauma, neuronal injury, neuronal toxicity, neuronal degeneration and stroke.

The present invention also provides for methods of treating stroke comprising administering an effective amount of a MuSK-activating agent to a subject in need of such treatment.

The present invention further provides for methods of identifying compounds that maintain or enhance memory or learning or enhance the recovery from a neurological impairment comprising screening for compounds that increase MuSK expression or stability in the brain, wherein an increase in MuSK in the presence of the compound compared to a control in which the compound was not present, indicates that the compound increases MuSK expression or stability. In one embodiment of the invention the increase in MuSK stability or expression is detected by the method selected from the group consisting of RT-PCR, Northern blot, immunohistochemistry, immunocytochemistry, RNase protection assay, immunoprecipitation, in situ hybridization, or Western blot analysis. In yet another embodiment of the invention, the screening for compounds that increase MuSK expression or stability is done in whole animals in vivo, in ex vivo explants of brain tissue or in cultured neurons.

The present invention also provides for methods of identifying compounds that enhance the recovery from a neurological impairment comprising screening for compounds that activate MuSK in the brain, wherein an increase in MuSK activity in the presence of the compound compared to a control in which the compound was not present, indicates that the compound increases MuSK activation. In one embodiment, the increase in MuSK activity is measured by detecting an increase in the phosphorylation of MuSK, an increase in agrin-MuSK binding or an increase in acetylcholinesterase receptor phosphorylation. The instant invention also comprises methods of treating a disease or condition associated with memory loss comprising administering an effective amount of a compound identified by these methods.

The present invention also provides for isolated nucleic acids comprising a nucleotide sequence that is at least 85% identical to the sequences selected from the group consisting of SEQ ID NO: 1, 20 and 18. In a further embodiment, the present invention provides for isolated polypeptides encoded for by an isolated nucleotide sequence that is at least 85% identical to the sequences selected from the group consisting of SEQ ID NO: 1, 20, and 18.

The present invention also provides for methods of treating a disease or condition associated with memory loss, such as Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, Parkinson's disease, and stroke, comprising administering an effective amount of Abgent antibody catalog # AP7664A to a subject in need of such treatment. In a particular embodiment of the invention, the condition associated with memory loss is Alzheimer's disease in its early stage or stroke in a mammal or a human. The invention further provides for methods of treating a neurological impairment, such as spinal cord injury, brain trauma, ischemia, shock, head trauma, neuronal injury, neuronal toxicity, neuronal degeneration and stroke, comprising administering an effective amount of Abgent antibody catalog # AP7664A to a subject in need of such treatment. In a particular embodiment of the invention, the neurological impairment is stroke in a mammal or a human.

The instant invention also comprises isolated nucleic acids comprising the nucleotide sequence set forth in SEQ ID NO: 18 and an isolated polypeptide encoded for by this nucleotide sequence.

### DESCRIPTION OF THE DRAWINGS

FIG. 1A is a graph demonstrating that MuSK is induced by IA training. It describes the results of quantitative real-time PCR (TaqMan), demonstrating that MuSK is induced by IA training. Data represent fold changes of MuSK mRNA in 20 − group versus 0 h− (control conditions) and in 20 h+ versus 0 h− (trained versus control). Values were normalized to those of glyceraldehyde-3-phosphate dehydrogenase (GAPDH). FIG. 1B is a graph showing the results of quantitated Western blot analyses of expression of the protein recognized by Abgent antibody catalog # AP7664A (San Diego, Calif.) of hippocampal extracts taken from control (0 h−, n=4), unpaired (n=4) and IA trained rats (20 h+, n=7). Values were normalized to those of GAPDH (B). Data are expressed as mean percentage±SEM of the 0 h− (100%) control mean values. Statistical analysis was performed using one-way ANOVA followed by Student-Newman-Keuls test. Trained animals showed a significant increase of MuSK mRNA and expression of the protein recognized by Abgent antibody catalog # AP7664A compared to 0 h− (*, p<0.05) and unpaired (p<0.05). No significant changes in MuSK mRNA or expression of the protein recognized by Abgent antibody catalog # AP7664A were found in unpaired compared with the 0h− group.

FIG. 2A is a schematic representation of brain MuSK sequence and PCR amplification fragments. The MuSK schematic is depicted with the signal sequence (SS), four Ig-like domains (Ig 1-IV), its C6 domain, and its kinase domain. Five overlapping PCR amplification fragments indicated between 5 sets of primers had been sequenced. from cDNAs obtained from hippocampus, cortex, cerebellum and hippocampal cultures. The 24 nucleotides and corresponding 8 amino acids that are present in muscle MuSK (GenBank accession U34985) but not the brain MuSK are shown in bold. FIG. 2B shows gel electrophoretic analysis of PCR amplifications performed with a set of primers that flanked the entire MuSK ORF and cDNAs obtained from hippocampus, cortex, cerebellum and hippocampal neuronal cultures (HNC). Two bands of 2,359 and 2,644 bp were generated. Their sequences revealed the existence of two alternatively spliced isoforms distinct for either presence or absence of an IgIII domain. FIG. 2C shows a schematic representation of the new MuSK short isoform expressed in the brain. The MuSK short isoform is depicted with its signal sequence (SS), three Ig-like domains, C6 and kinase domains. This shorter isoform was characterized by the A₄₅₄ substitution and a deletion of the IgIII domain (ΔIgIII). The nucleotide sequence of this shorter isoform is set forth in SEQ ID NO: 18 and its amino acid sequence is set forth in SEQ ID NO: 19. FIG. 2D shows a Western blot analysis of hippocampal cell cultures, adult and post-natal day 1 tissues. Western blot immunostaining of 25 μg of total protein extract from the indicated tissues and cell cultures shows the relative concentration of the protein recognized by Abgent antibody catalog # AP7664A (San Diego, Calif.).

FIG. 3A is a timeline of the anti-MuSK antisense oligonucleotide (ODN) injection experiments (injection, training and testing points are shown). FIG. 3B demonstrates that MuSK antisense ODN blocks IA memory retention. Mean latency of IA acquisition (Acq) and memory retention (Test) expressed in seconds (s). Hippocampal double-injection of MuSK antisense (MuSK-ODN) immediately after and 8 h following IA training (n=4) significantly blocks memory retention at 24 h compared to double injection of scrambled-ODN (Sc-ODN) (n=4, *p<0.05) or PBS. Because both Sc-ODN and PBS injected-groups had similar latencies, they were combined. FIGS. 3C-3E show that hippocampal disruption of MuSK affects CREB phosphorylation. FIG. 3C shows injection and training time points. Quantitative Western blot analysis of hippocampal extracts from trained rats that received intrahippocampal injection of MuSK-ODN (n=8) or Sc-ODN (n=8) one hour before training and sacrificed 4 hours later (FIG. 3D). Blots were stained with anti-pCREB (Ser-133) antibody stripped and re-stained with anti-CREB, anti-NP62 antibodies and finally with anti-actin, which was used for normalization. Four representative samples per condition are shown in FIG. 3D. FIG. 3E shows graphs representing the densitometric analysis of all data. Data are expressed as a mean percentage±SEM of the Sc-ODN (100%) control group. FIGS. 3F-G show that MuSK antisense ODN does no affect acquisition or short-term memory. FIG. 3F shows injection, training and testing time points. FIG. 3G shows mean latency±SEM of IA acquisition (Acq) and memory retention test at 1 and 24 hours after IA training, expressed in second(s). Hippocampal double-injection of MuSK-ODN 14 and 6 hours before IA training (n=8) does not block memory retention at 1 h compared to double injection of Sc-ODN performed at the same time points (n=8). Retention test 24 hours after training revealed significant impairment in MuSK-ODN injected rats (n=8, *p<0.05) compared to its control group (Sc-ODN, n=8).

FIG. 4A is a timeline of antibody (Abgent antibody cat # AP7664A, San Diego, Calif.) injection experiments (injection, training and testing points are shown). FIG. 4B is a graph demonstrating that Abgent antibody cat # AP7664A injection enhances IA retention. Mean latency of IA acquisition (Acq) and memory retention (Test) are expressed in seconds (s). Hippocampal injection of Abgent antibody cat # AP7664A immediately after IA training (n=8) significantly enhances memory retention at 24 h compared to IgG or PBS injection (***p<0.001). Because both IgG and PBS injections produced similar retention latencies, the behavioral data of the two groups have been combined (IgG, n=5 and PBS, n=3). The unpaired (UNP) group, as expected, showed no memory retention.

FIG. 5A is a graph showing the results of quantitated Western blot analysis, demonstrating three-day treatment of cultured neurons with MuSK-ODN, scrambled-ODN (Sc-ODN) and untreated (control). The graph shows that cultured neurons treated with MuSK-ODN but not with Sc-ODN reduces the level of the protein recognized by Abgent antibody catalog # AP7664A (San Diego, Calif.) compared to the control. FIG. 5B shows microscope images demonstrating that MuSK antisense oligodeoxynucleotide (ODN) treatment induces morphological changes in primary hippocampal neurons. The knock down of MuSK expression in cultured hippocampal neurons resulted in reduced branching of both axons and dendrites and abnormal elongation of the axonal processes (Tau panels were stained with anti-Tau antibodies to mark axonal markers and Map2 panels were stained with anti-Map2 antibodies to mark dendritic markers).

FIGS. 6A-J show that membrane expressed MuSK co-localizes with nicotinic acetylcholine receptors (AChRs). Double immunostainings of hippocampal neuronal cultures with: (A) MuSK/α-bungarotoxin (α-Btx); (B) MuSK/GABA_{A} receptor β(GABA_{A}); (C) MuSK/GluR1; (D) MuSK/muscarinic acetylcholine receptor (mAChR); (E) MuSK/PSD-93; (F) MuSK/PSD-95; (G) MuSK/agrin; (H) MuSK/synapsin; (I) MuSK/MAP2 and (J) MuSK/Tau. Note that membrane-expressed MuSK largely co-localized with α-Btx and PSD-93 but rarely with mAChR, GABA, GluR1 and PSD-95. MuSK co-localization with agrin is only marginal. Double staining of MuSK/synapsin indicated synaptic localization and double staining of MuSK with MAP2 or TAU revealed that MuSK is mostly distributed on the dendrites. Scale bars: Panel a of A-J-25 μm, panel b-d of A-J −2.5 μm.

FIG. 7 shows that agrin treatment of hippocampal neuronal cultures (HNC) results in increased membrane expression of MuSK. Quantitative morphometric data obtained from the membrane expressed MuSK immunostainings with or without agrin treatment. Data are expressed as % of Control (100%). Agrin treatment resulted in significant increase in MuSK membrane expression at 4h but not at earlier time points.

FIGS. 8A and B are graphs demonstrating that agrin is induced by IA training. Northern (A) and Western (B) blot analyses of hippocampal extracts taken from control (0 h−, n=4), unpaired (n=4) and IA trained rats (20 h+, n=7). Values were normalized to cyclophilin (A) or GAPDH (B). Data are expressed as mean percentage±SEM of the 0h− (100%) control mean values. Statistical analysis was performed using one-way ANOVA followed by Student-Newman-Keuls test. Trained animals showed a significant increase of agrin mRNA and protein levels compared to 0 h− (*, p<O.05) and unpaired (p<0.05). No significant changes in agrin were found in the unpaired compared with the 0 h− group.

FIG. 9 is a graph demonstrating that delivery of Abgent antibody cat # AP7664A into the cerebral cortex after focal ischemia enhances motor recovery. Adult male Long-Evans rats were given focal infarctions within forelimb motor cortex. Half of the animals then received Abgent antibody cat # AP7664A (n=5) or vehicle (n=5) into the damaged cortex. Skilled reaching ability on the single pellet reaching task was monitored daily after injury (Kleim et al., (2003) Neurological Research, 25: 789-793). A one-way repeated measures ANOVA showed a significant Time×Treatment interaction (p<0.05) where Abgent antibody cat # AP7664A-injected animals exhibited significantly greater reaching accuracies as training progressed (*Fishers PLSD; p<0.05).

### DETAILED DESCRIPTION

The present invention concerns the discovery that MuSK is expressed in the brain and that MuSK expression in the brain increases with learning and memory. The inventors have discovered that MuSK is a component of the molecular signaling pathway for learning and memory and is involved in normal connectivity formation in the brain.

The present invention also concerns the discovery that treatment with Abgent antibody cat # AP7664A, (San Diego, Calif.) in the brain enhances learning and memory and recovery from stroke. The protein recognized by this antibody is expressed in the brain and provides a further component in the pathway of learning and memory.

In the adult mammalian brain, the hippocampus is known to mediate the consolidation of contextual and spatial memories. To identify genes that are regulated during memory consolidation, DNA microarray hybridizations were screened for genes differentially expressed in the hippocampus after inhibitory avoidance learning. MuSK was identified as a gene that is differentially expressed during memory consolidation. Knocking down MuSK expression decreased memory retention, confirming that MuSK is required for memory formation.

The present invention also teaches the activation of MuSK to enhance memory and learning. Thus, the present invention provides for methods of activating MuSK or increasing MuSK expression in order to treat diseases or conditions associated with memory loss such as Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, Parkinson's disease, and stroke.

The present invention also teaches that, although required for consolidation of long-term memory, MuSK is not required for memory acquisition or short term memory. Furthermore, one possible mechanism by which MuSK acts, via the cAMP response element binding protein (CREB) pathway. Accordingly, the present invention involves the use of MuSK to activate the CREB pathway in order to enhance memory or protect from memory disorders such as Alzheimer's disease.

The present invention is also concerned with the discovery that MuSK is essential for proper synapse formation. Thus, the present invention also provides for methods of activating MuSK or increasing MuSK expression in order to treat conditions associated with neurological impairment and damage such as spinal cord injury, brain trauma, ischemia, shock, head trauma, neuronal injury, neuronal toxicity, neuronal degeneration and stroke.

The discovery that MuSK is essential for learning, memory and the formation of morphologically normal synapses makes MuSK a particularly attractive target for the treatment of stroke. The extensive repetitive training stroke patients undergo in order to regain function likely involves the remodeling and reformation of synapses (Tully et al., (2003) Nature Reviews 2: 267-277). Thus, administration of a MuSK-activating agent in conjunction with rehabilitative training can result in enhanced recovery from stroke. Further, administration of Abgent antibody cat # AP7664A, (San Diego, Calif.) in conjunction with rehabilitative training, as shown in Example 8, also provides enhanced recovery from stroke.

The present invention provides for methods of maintaining or enhancing memory or learning in a mammal comprising activating MuSK or increasing the expression of MuSK in the brain. In one embodiment, MuSK is activated or MuSK expression is increased in the hippocampus, the area of the brain known to mediate contextual and spatial memories. In a further embodiment, MuSK is activated in one or more of the following regions of the brain in which MuSK mRNA was detected: the hippocampus, amygdala, cortical region and/or cerebellum.

In one embodiment of the invention, the type of memory enhanced by MuSK activation or increased expression is long-term memory. In a further embodiment, the type of memory enhanced is working memory. In yet another embodiment, the type of memory enhanced is memory consolidation.

In one embodiment of the present invention, MuSK is activated by a MuSK-activating antibody. In this embodiment of the invention, the activating antibody is any antibody that activates MuSK. Such activating antibodies will be appreciated by those of ordinary skill in the art because they increase MuSK activity (as measured, for example, by MuSK phosphorylation) and are specific for the amino terminus (extracellular domain) of MuSK. Examples of antibodies recognizing the amino terminus (extracellular domain) of MuSK, and thus potential MuSK-activating antibodies, are Affinity Bioreagent's rabbit anti-extracellular domain (a.a. 210-304) MuSK antibody (Golden, Colo.; cat # pA1-1741) and R&D System's goat anti-MuSK antibody (Minneapolis, Minn.; cat # AF562). Another antibody that may be used in the present invention is Abgent antibody cat # AP7664A (San Diego, Calif.).

In yet other embodiments of the invention, the MuSK-activating agent is a compound identified to increase MuSK expression or activity (a MuSK agonist) or is an inhibitor of a MuSK inhibitor (an inhibitor of a MuSK antagonist). In yet another embodiment, MuSK expression is increased by stabilizing or preventing the degradation of MuSK polypeptide or mRNA.

The present invention also provides for methods of maintaining or enhancing memory or learning in a mammal comprising increasing MuSK expression in the brain. In one preferred embodiment of the invention, MuSK expression is increased by delivering a nucleotide sequence encoding MuSK to the brain of the animal by means of e.g. gene therapy. Delivery of the MuSK polypeptide outside of its transmembrane context is not desired because such a polypeptide would act to downregulate endogenous MuSK function.

The present invention concerns the discovery that MuSK is expressed in the brain and the description of two rat brain MuSK sequences (whose nucleotide sequences are depicted in SEQ ID NO: 1 and 20 (SEQ ID NO: 1 includes the coding sequence of the rat brain MuSK isoform with the A₄₅₄ substitution and 5′ and 3′ non-coding sequences, while SEQ ID NO: 20 represents the coding sequence of the rat brain MuSK isoform with the A₄₅₄ substitution (and no 5′ or 3′ sequences), and SEQ ID NO: 18, which represents the coding sequence of the rat brain isoform carrying both the A₄₅₄ substitution and ΔIgIII (an isoform carrying both the A₄₅₄ substitution and ΔIgIII has not previously been described) and whose amino acid sequences are depicted in SEQ ID NO: 2 and 19, respectively). The present invention, however, is not limited to the upregulation and/or activation of rat brain MuSK. Any brain MuSK can be upregulated and/or activated and one of ordinary skill in the art would readily be able to identify, detect and measure the activity of MuSK in the brain of other species.

The present invention comprises isolated nucleic acid sequences that are at least 85% identical to the sequences selected from the group consisting of SEQ ID NO: 1, 20 and 18. In a preferred embodiment the present invention comprises isolated nucleic acid sequences that are at least 90% identical the sequences selected from the group consisting of SEQ ID NO: 1, 20 and 18. In a more preferred embodiment, the isolated nucleic acid is 95% or 99% identical the sequences selected from the group consisting of SEQ ID NO: 1, 20 and 18. In yet a more preferred embodiment the isolated nucleic acid comprises SEQ ID NO: 1, SEQ ID NO: 20, or SEQ ID NO: 18.

The present invention also comprises polypeptides encoded for by the above-described nucleic acid sequences. In one embodiment, the polypeptide is at least 93% identical to SEQ ID NO: 2 or SEQ ID NO: 19. In a more preferred embodiment the polypeptide is at least 95% or 99% identical to SEQ ID NO: 2 or SEQ ID NO: 19. In a yet more preferred embodiment, the polypeptide comprises SEQ ID NO: 2 or SEQ ID NO: 19.

The present invention provides for methods of treating a disease or condition associated with memory loss comprising administering an effective amount of a MuSK-activating agent to a subject in need of such treatment. In one preferred embodiment, such a condition is stroke.

In yet another embodiment of the invention, the condition associated with memory loss that is treated with a MuSK-activating agent is Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, or Parkinson's disease. In a preferred embodiment, the condition is Alzheimer's disease or senile dementia of the Alzheimer's type.

The present invention also provides for methods of treating a neurological impairment comprising administering an effective amount of a MuSK-activating agent to a subject in need of such treatment. Such conditions include spinal cord injury, brain trauma, ischemia, shock, head trauma, neuronal injury, neuronal toxicity, and neuronal degeneration.

The identification of MuSK as critical to the formation, maintenance and retention of memories indicates that compounds that enhance MuSK activation in the brain can be used to maintain or enhance learning or memory, such as in patients with conditions associated with memory loss. An advantage of such compounds is that they may not need to be capable of entering the cell, due to the possibility of activating MuSK via its extracellular domain. Examples of such conditions include Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, Parkinson's disease, and stroke. Furthermore, such compounds can be used to treat conditions associated with general neurological impairment such as spinal cord injury and stroke. Thus, the present invention provides for methods of identifying compounds that activate MuSK in the brain. In one embodiment, activation of MuSK is detected by measuring an increase in MuSK phosphorylation. In yet other embodiments, activation of MuSK is detected by measuring an increase in other downstream indicators of MuSK activation, such as increased MuSK-agrin binding, increased acetylcholinesterase receptor phosphorylation, increased CREB phosphorylation, and increased presynaptic and postsynaptic differentiation.

Thus, the present invention provides for methods of identifying compounds that increase MuSK expression in the brain. In one embodiment, such increased expression is detected by measuring an increase in MuSK mRNA levels. An increase in MuSK mRNA levels can be detected, for example, by RT-PCT, Northern blot, RNase protection assay or in situ hybridization. In yet another embodiment of the invention, such increased expression is detected by measuring an increase in MuSK polypeptides levels. An increase in MuSK polypeptide levels can be achieved, for example, by immunohistochemistry, immunocytochemistry, immunoprecipitation or Western blot analysis.

The above described screening experiments can be done in the whole animal in vivo, in ex vivo explants of brain tissue or in cultured cells, for example cultured neurons or in cell lines that model neuronal cells.

### Delivery

The MuSK-activating agents of the present invention and the agents or compounds that increase MuSK expression may be delivered to the animal being treated or tested via intralesional, intramuscular or intravenous injection; infusion; liposome mediated delivery; viral infection; gene bombardment; topical, nasal, oral, anal, ocular, cerebro-spinal, or otic delivery.

According to the invention, a therapeutic compound can be formulated in a pharmaceutical composition to be introduced parenterally, transmucosally, e.g., orally, nasally, or rectally, or transdermally. Preferably, administration is parenteral, e.g., via intravenous injection, and also including, but is not limited to, intra-arteriole, intramuscular, intradermal, subcutaneous, intraperitoneal, intraventricular, and intracranial administration.

In another embodiment, the therapeutic compound can be delivered in a vesicle, in particular a liposome (see Langer, Science 1990; 249:1527-1533; Treat et al., in Liposomes in the Therapy of Infectious Disease and Cancer, Lopez-Berestein and Fidler (eds.), New York: Liss, 1989, pp. 353-365; Lopez-Berestein, ibid., pp. 317-327; see generally ibid.). To reduce its systemic side effects, this may be a preferred method for introducing the therapeutic compound.

In yet another embodiment, the therapeutic compound can be delivered in a controlled release system. For example, a compound may be administered using intravenous infusion with a continuous pump, in a polymer matrix such as poly-lactic/glutamic acid (PLGA), a pellet containing a mixture of cholesterol and the therapeutic compound (SilasticR™; Dow Coming, Midland, Mich.; see U.S. Pat. No. 5,554,601) implanted subcutaneously, an implantable osmotic pump, a transdermal patch, liposomes, or other modes of administration. In one embodiment, a pump may be used (see Langer, supra; Sefton, CRC Crit. Ref. Biomed. Eng. 1987; 14:201; Buchwald et al., Surgery 1980; 88:507; Saudek et al., N. Engl. J. Med. 1989; 321:574). In another embodiment, polymeric materials can be used (see Langer and Wise (eds.), Medical Applications of Controlled Release, Boca Raton, Fla.: CRC Press, 1974; Controlled Drug Bioavailability, Drug Product Design and Performance, Smolen and Ball (eds.), New York: Wiley, 1984; Ranger and Peppas, J. Macromol. Sci. Rev. Macromol. Chem. 1983, 23:61; see also Levyet al., Science 1985, 228:190; During et al., Ann. Neurol. 1989, 25:351; Howard et al., J. Neurosurg. 1989, 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, 1984, pp. 115-138). Preferably, in a subject suffering from brain amyloidosis, a condition associated with Alzheimer's disease, a controlled release device is introduced into a subject in proximity of the site of amyloidosis. Other controlled release systems are discussed in the review by Langer (Science 1990, 249:1527-1533).

A constant supply of the therapeutic compound can be ensured by providing a therapeutically effective dose (i.e., a dose effective to induce metabolic changes in a subject) at the necessary intervals, e.g., daily, every 12 hours, etc. These parameters will depend on the severity of the disease condition being treated, other actions, such as diet modification, that are implemented, the weight, age, and sex of the subject, and other criteria, which can be readily determined according to standard good medical practice by those of skill in the art.

### Subjects of Administration

A subject in whom administration of the therapeutic compound is an effective therapeutic regiment for a disease or disorder is preferably a human, but can be any animal, including a laboratory animal in the context of a clinical trial or screening or activity experiment. Thus, as can be readily appreciated by one of ordinary skill in the art, the methods and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, cows, and non-human primates etc., avian species, such as chickens, turkeys, songbirds, etc., i.e., for veterinary medical use.

### Injectable Delivery

The methods according to this invention can be achieved via injectable administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

### Penetrating the Blood Brain Barrier

Because the present invention relates to methods of treating neurological-associated conditions and for identifying activators of MuSK in the brain, it will often be necessary for the agents of the present invention to penetrate the blood brain barrier.

The blood brain barrier of subjects suffering from brain amyloidosis, which is associated with Alzheimer's disease, is often found in deteriorated condition. This deteriorated condition of the blood brain barrier facilitates the ability of agents administered parenterally to traverse the barrier.

For CNS administration, a variety of techniques are available for promoting transfer of a therapeutic agent across the blood brain barrier, including disruption by surgery or injection, co-administration of a drug that transiently opens adhesion contacts between CNS vasculature endothelial cells, and co-administration of a substance that facilitates translocation through such cells.

The therapeutic agents of the present invention can be administered directly to the brain or cerebrospinal fluid, e.g., by direct cranial or intraventricular injection, or may pass through the blood brain barrier following administration by parenteral injection, oral administration, skin absorption, etc.

In another embodiment, the agents of the present invention can be conjugated with a targeting molecule, such as transferrin, for which there are receptors on the blood brain barrier. Another such targeting method is using nanogel (cross-linked poly(ethylene glycol) and polyethylenimine) modified with specific targeting molecules to deliver oligonucleotides to the brain (Vinogradov et al., (2004) Bioconjug Chem. 15(1): 50-60). In a further embodiment, the agents of the present invention can be modified to have decreased polarity, or increased hydrophobicity, as more hydrophobic (less polar) agents cross the blood brain barrier more readily. In yet another embodiment, the agents of the present invention can be administered in a liposome, particularly a liposome targeted to the blood brain barrier. Administration of pharmaceutical agents in liposomes is known (see Langer, 1990, Science 249:1527-1533; Treat et al., in Liposomes in the Therapy of Infectious Disease and Cancer, Lopez-Berestein and Fidler (eds.), Liss, New York, pp.353-365 (1989); Lopez-Berestein, ibid., pp. 317-327; see generally ibid.).

Another way to penetrate the blood-brain barrier is small heterocyclic organic molecules. The present invention includes activating MuSK in the brain of a subject by using small heterocyclic organic molecules, such molecules being capable of penetrating the blood brain barrier.

These and other strategies for directing therapeutic agents across the blood brain barrier are known in the art, and contemplated by the present invention.

### Dosages

For all of the compounds and agents delivered using the methods of this invention, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. The dosing schedule may vary, depending on the circulation half-life, and the formulation used.

Toxicity and therapeutic efficacy of compounds can be determined by standard pharmaceutical procedures, for example in cell culture assays or using experimental animals to determine the LD50 and the ED50. The parameters LD50 and ED50 are well known in the art, and refer to the doses of a compound that are lethal to 50% of a population and therapeutically effective in 50% of a population, respectively. The dose ratio between toxic and therapeutic effects is referred to as the therapeutic index and may be expressed as the ratio: LD50/ED50. Compounds that exhibit large therapeutic indices are preferred. While compounds that exhibit toxic side effects may be used. However, in such instances it is particularly preferable to use delivery systems that specifically target such compounds to the site of affected tissue (e.g. the brain and other neuronal tissues) so as to minimize potential damage to other cells, tissues or organs and to reduce side effects.

Data obtained from cell culture assay or animal studies may be used to formulate a range of dosages for use in humans. For example, in the present invention 1 μl of a 250 ng/μl solution of Abgent antibody cat # AP7664A, (San Diego, Calif.) was injected directly into the brain of the rat. A comparable amount to deliver directly to the human brain, taking into account the larger size of the human brain compared to the rat brain, would be 1 ml of the 250 ng/μl MuSK-activating antibody solution. Systemic administration of MuSK-activating antibody conjugated to a molecule that allows for the antibody to cross the blood-brain barrier would be in the dosage range of approximately 1-5 mg/kg body weight. Preferably, systemic administration of MuSK-activating antibody would be in the dose of approximately 3 mg/kg body weight. The dosage of compounds used in therapeutic methods of the present invention preferably lie within a range of circulating concentrations that includes the ED50 concentration but with little or no toxicity (e.g., below the LD50 concentration). The particular dosage used in any application may vary within this range, depending upon factors such as the particular dosage form employed, the route of administration utilized, the conditions of the individual (e.g., patient), and so forth.

A therapeutically effective dose may be initially estimated from cell culture assays and formulated in animal models to achieve a circulating concentration range that includes the IC50. The IC50 concentration of a compound is the concentration that achieves a half-maximal inhibition of symptoms (e.g., as determined from the cell culture assays). Appropriate dosages for use in a particular individual, for example in human patients, may then be more accurately determined using such information.

Measures of compounds in plasma may be routinely measured in an individual such as a patient by techniques such as high performance liquid chromatography (HPLC) or gas chromatography.

The MuSK-activating agents of the present invention (or their derivatives) may be administered in conjunction with one or more additional active ingredients or pharmaceutical compositions.

### Screening and Chemistry

The present invention contemplates methods for identifying agonists of MuSK and antagonists of MuSK inhibitors, as well as compounds that increase the expression of MuSK mRNA and/or polypeptide. Such agonists and antagonists and agents that increase MuSK expression are referred to herein as “compounds.” Compounds can be lead compounds for further development, or therapeutic candidates for pre-clinical and clinical testing.

Any screening technique known in the art can be used to screen for agonists or antagonists. The present invention contemplates screens for small molecules and mimics, as well as screens for natural products that bind to and agonize MuSK. For example, natural products libraries can be screened using assays of the invention for molecules that agonize MuSK activity in the brain.

Knowledge of the primary sequence of MuSK inhibitors and MuSK, can provide an initial clue as what compounds might be agonists of MuSK function or antagonists of MuSK inhibitors. Identification and screening of agonists and antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

Another approach for identifying MuSK agonists or MuSK inhibitor-antagonists uses recombinant bacteriophage to produce large libraries. Using the “phage method” (Scott and Smith, Science 1990, 249:386-390; Cwirla, et al., Proc. Natl. Acad. Sci. USA 1990, 87:6378-6382; Devlin et al., Science 1990, 49:404-406), very large libraries can be constructed (106-108 chemical entities). A second approach uses primarily chemical methods, of which the Geysen method (Geysen et al., Molecular Immunology 1986, 23:709-715; Geysen et al. J. Immunologic Methods 1987, 102:259-274; and the method of Fodor et al. (Science 1991, 251:767-773) are examples. Furka et al. (14th International Congress of Biochemistry 1988, Volume #5, Abstract FR:013; Furka, Int. J. Peptide Protein Res. 1991, 37:487-493), Houghton (U.S. Pat. No. 4,631,211) and Rutter et al. (U.S. Pat. No. 5,010,175) describe methods to produce a mixture of peptides that can be tested as agonists or antagonists.

In another aspect, synthetic libraries (Needels et al., Proc. Natl. Acad. Sci. USA 1993, 90:10700-4; Ohlmeyer et al., Proc. Natl. Acad. Sci. USA 1993, 90:10922-10926; Lam et al., PCT Publication No. WO 92/00252; Kocis et al., PCT Publication No. WO 9428028) and the like can be used to screen for compounds to be used in the methods according to the present invention.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (NC), or are readily producible. Additionally, natural and synthetically produced libraries and compounds are readily modified through conventional chemical, physical, and biochemical means (Blondelle et al., TIBTech 1996, 14:60).

### High-Throughput Screening

The screening methods of the present invention may performed using high-throughput assays, including without limitation cell-based or cell-free assays. It will be appreciated by those skilled in the art that different types of assays can be used to detect different types of agents. Several methods of automated assays have been developed in recent years so as to permit screening of tens of thousands of compounds in a short period of time (see, e.g., U.S. Pat. Nos. 5,585,277, 5,679,582, and 6,020,141). Alternatively, simple reporter-gene based cell assays can be used.

### In Vivo Screening Methods

Intact cells, explants of tissue from an animal, or whole animals expressing a gene encoding MuSK can be used in screening methods to identify candidate compounds.

In one series of embodiments, a permanent cell line is established. Alternatively, cells are transiently programmed to express MuSK by introduction of appropriate DNA or mRNA, e.g., using the vector systems described above. In still another embodiment, hippocampal neural cells, which express MuSK endogenously, can be used. Identification of candidate compounds can be achieved using any suitable assay, including without limitation (i) assays that measure selective binding of test compounds to MuSK and (ii) assays that measure the ability of a test compound to modify (i.e., inhibit or enhance) a measurable activity or function of MuSK, e.g. inhibitory avoidance training. In another embodiment of the invention, the compound is tested in the whole animal.

A “test compound” is a molecule that can be tested for its ability to act as a modulator of a gene or gene product. Test compounds can be selected without limitation from small inorganic and organic molecules (i.e., those molecules of less than about 2 kD, and more preferably less than about 1 kD in molecular weight), polypeptides (including native ligands, antibodies, antibody fragments, and other immunospecific molecules), oligonucleotides and polynucleotide molecules. In various embodiments of the present invention, a test compound is tested for its ability to modulate the expression or stability of a MuSK-encoding nucleic acid or MuSK protein or bind to a MuSK protein. A compound that modulates a nucleic acid or protein of interest is designated herein as a “candidate compound” or “lead compound” suitable for further testing and development. Candidate compounds include, but are not necessarily limited to, the functional categories of agonists.

An “agonist” is defined herein as a compound that interacts with (e.g., binds to) a nucleic acid molecule or protein, and promotes, enhances, stimulates or potentiates the biological expression or function of the nucleic acid molecule or protein. The term “partial agonist” is used to refer to an agonist that interacts with a nucleic acid molecule or protein, but promotes only partial function of the nucleic acid molecule or protein. A partial agonist may also inhibit certain functions of the nucleic acid molecule or protein with which it interacts. An “antagonist” interacts with (e.g., binds to) and inhibits or reduces the biological expression or function of the nucleic acid molecule or protein.

### Gene Therapy

In a specific embodiment, vectors comprising a sequence encoding MuSK are administered to treat or prevent a disease, disorder or condition associated with memory loss and/or to enhance recovery from stroke.

Any of the methods for gene therapy available in the art can be used according to the present invention. Exemplary methods are described below. For general reviews of the methods of gene therapy, see, Goldspiel et al., Clinical Pharmacy 1993, 12:488-505; Wu and Wu, Biotherapy 1991, 3:87-95; Tolstoshev, Rev. Pharmacol. Toxicol. 1993, 32:573-596; Mulligan, Science 1993, 260:926-932; and Morgan and Anderson, Ann. Rev. Biochem. 1993, 62:191-217; May, TIBTECH 1993, 11:155-215. Methods commonly known in the art of recombinant DNA technology that can be used are described in Ausubel et al. (eds.), Current Protocols in Molecular Biology, New York: John Wiley & Sons, 1993; Kriegler, Gene Transfer and Expression, A Laboratory Manual, New York: Stockton Press, 1990; and Dracopoli et al. (eds.), Current Protocols in Human Genetics, New York: John Wiley & Sons, 1994, chapters 12-13. Vectors suitable for gene therapy are described herein.

In one embodiment, a vector is used in which the coding sequences and any other desired sequences are flanked by regions that promote homologous recombination at a desired site in the genome, thus providing for expression of the construct from a nucleic acid molecule that has integrated into the genome (Koller and Smithies, Proc. Natl. Acad. Sci. USA 1989, 86:8932-8935; Zijlstra et al., Nature 1989, 342:435-438).

Delivery of the vector into a patient may be either direct, in which case the patient is directly exposed to the vector or a delivery complex, or indirect, in which case, cells are first transformed with the vector in vitro, then transplanted into the patient. These two approaches are known, respectively, as in vivo and ex vivo gene therapy.

In a specific embodiment, the vector is directly administered in vivo, where it enters the cells of the organism and mediates expression of the construct. This can be accomplished by any of numerous methods known in the art and discussed above, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (poly-β-1-→4-N-acetylglucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem. 1987, 62:4429-4432), etc. In another embodiment, a nucleic acid-ligand complex can be formed in which the ligand comprises a fusogenic viral peptide to disrupt endosomes, allowing the nucleic acid to avoid lysosomal degradation, or cationic 12-mer peptides, e.g., derived from antennapedia, that can be used to transfer therapeutic DNA into cells (Mi et al., Mol. Therapy 2000, 2:339-47). In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Additional targeting and delivery methodologies are contemplated in the description of the vectors, below.

Preferably, for in vivo administration of viral vectors, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors (see, e.g., Wilson, Nature Medicine 1995). In that regard, it is advantageous to employ a viral vector that is engineered to express a minimal number of antigens.

### Vectors

Preferred vectors in vitro, in vivo, and ex vivo are viral vectors, such as lentiviruses, retroviruses, herpes viruses, adenoviruses, adeno-associated viruses, vaccinia virus, baculovirus, alphavirus, influenza virus, and other recombinant viruses with desirable cellular tropism. In one embodiment of the invention, lentiviral vectors are preferred because of the lentiviral characteristic of being able to infect non-dividing cells such as neurons.

A gene encoding a functional or mutant protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication WO 95/28494.

Viral vectors commonly used for in vivo or ex vivo targeting and therapy procedures are DNA virus vectors and RNA virus vectors. Methods for constructing and using viral vectors are known in the art (see, e.g., Miller and Rosman, BioTechniques 1992, 7:980-990). Preferably, the viral vectors are replication defective, that is, they are unable to replicate autonomously, and thus are not infectious, in the target cell. Preferably, the replication defective virus is a minimal virus, i.e., it retains only the sequences of its genome which are necessary for encapsidating the genome to produce viral particles. Defective viruses, which entirely or almost entirely lack viral genes, are preferred. Use of defective viral vectors allows for administration to cells in a specific, localized area, without concern that the vector can infect other cells. Thus, a specific tissue can be specifically targeted.

### Non-Viral Vectors

In another embodiment, the vector can be introduced in vivo by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. USA 1987, 84:7413-7417; Felgner and Ringold, Science 1989, 337:387-388; see Mackey, et al., Proc. Natl. Acad. Sci. USA 1988, 85:8027-8031; Ulmer et al., Science 1993, 259:1745-1748). Useful lipid compounds and compositions for transfer of nucleic acids are described in PCT Publication Nos. WO 95/18863 and WO96/17823, and in U.S. Pat. No. 5,459,127. Lipids may be chemically coupled to other molecules for the purpose of targeting (see Mackey, et. al., supra). Targeted peptides, e.g., hormones or neurotransmitters, and proteins such as antibodies, or non-peptide molecules could be coupled to liposomes non-covalently as well, by insertion via a membrane binding domain or segment into the bilayer membrane.

Other molecules are also useful for facilitating transfection of a nucleic acid in vivo, such as a cationic oligopeptide (e.g., PCT Publication No. WO95/21931), peptides derived from DNA binding proteins (e.g., PCT Publication No. WO96/25508), or a cationic polymer (e.g., PCT Publication No. WO95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (see, e.g., Wu et al., J. Biol. Chem. 1992, 267:963-967; Wu and Wu, J. Biol. Chem. 1988, 263:14621-14624; Canadian Patent Application No. 2,012,311; Williams et al., Proc. Natl. Acad. Sci. USA 1991, 88:2726-2730). Receptor-mediated DNA delivery approaches can also be used (Curiel et al., Hum. Gene Ther. 1992, 3:147-154; Wu and Wu, J. Biol. Chem. 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci. 1998, 321:893; PCT Publication Nos. WO 99/01157, WO 99/01158, and WO 99/01175).

Definitions

As used herein the term “memory” means the capability to retain the knowledge of previous thoughts, impressions and events.

As used herein the term “long term memory” means a memory that lasts for more than one or two minutes.

As used herein the term “working memory” means memory for intermediate results that must be held during thinking.

As used herein the term “memory consolidation” means the process by which learned information is transformed into stable modifications.

The term “stroke” as used herein is the acute onset of neurological deficit. Ischemic stroke (focal ischemia), the most common type of stroke, may be defined as a focal loss of brain tissue that results from insufficient blood supply to a particular brain area, usually as a consequence of an embolus, thrombi, or local atheromatous closure of the blood vessel. The loss of brain tissue induces a variety of neurological deficits including amnesia, aphasia, and hemiparesis.

The phrase “therapeutically effective amount” is used herein to mean an amount sufficient to reduce by at least about 15 percent, preferably by at least 50 percent, more preferably by at least 90 percent, and most preferably prevent, a clinically significant deficit in the activity, function and response of the host. Alternatively, a therapeutically effective amount is sufficient to cause an improvement in a clinically significant condition in the host.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “subject in need thereof” as used herein refers to a mammal. In particular, the term refers to humans diagnosed with Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, Parkinson's disease, spinal cord injury, brain trauma, ischemia, shock, head trauma, neuronal injury, or neuronal toxicity. In a preferred embodiment, the term refers to a mammal diagnosed with having suffered from a stroke.

The term “treat” is used herein to mean to relieve or alleviate at least one symptom of a disease in a subject. For example, in relation to dementia, the term “treat” may mean to relieve, alleviate or delay the onset of cognitive impairment (such as impairment of memory and/or orientation) or impairment of global functioning (activities of daily living). Examples of tests that can be used to assess the memory of a human or to assess the recovery of a human from neurological impairment include the Wechsler memory scale-revised, the Rey complex figure test, the short recognition memory test for words and faces, the doors and people test, the camel and cactus test, the concrete and abstract work synonym test, the graded naming test, the Della Sala et al.'s dual task performance test (Della Sala (1995) Ann NY Acad Sci 769:161-171), the Stroop task, the Wisconsin card sorting test, the test of everyday attention, the visual object and space perception battery. Each of these tests is described in Graham et al. ((2004) J Neurol Neurosurg Psychiatry 75:61-71).

In relation to stroke, the term “treat” may mean to enhance motor performance, cognition, memory and every day living skills as measured by standard stroke assessment scales including the NIH Stroke Scale, Fugl-Meyer, Scandinavian Stroke Supervision, Barthel and Mathew scales (Roden-Jullig et al., (1994) J. Intern. Med. 236(2): 125-136; Hellstrom et al., (2003) J. Rehabil. Med. 35(5): 202-207).

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. Numerical quantities given herein are approximate unless stated otherwise, meaning that the term “about” or “approximately” can be inferred when not expressly stated.

“Sequence alignment” means the process of lining up two or more sequences to achieve maximal levels of sequence identity (and, in the case of amino acid sequences, conservation), e.g., for the purpose of assessing the degree of sequence similarity. Numerous methods for aligning sequences and assessing similarity and/or identity are known in the art such as, for example, the Cluster Method, wherein similarity is based on the MEGALIGN algorithm, as well as BLASTN, BLASTP, and FASTA (Lipman and Pearson, 1985; Pearson and Lipman, 1988). When using all of these programs, the preferred settings are those that result in the highest sequence similarity.

Molecular Biology

In accordance with the present invention there may be employed conventional molecular biology, microbiology, cell culture, protein expression and purification, antibody, and recombinant DNA techniques within the skill of the art. Such techniques are explained fully in the literature. See, e.g., Sambrook, Fritsch and Maniatis, Molecular Cloning: A Laboratory Manual, Second Edition (Cold Spring Harbor Laboratory Press, New York: 1989); DNA Cloning: A Practical Approach, Volumes I and II (Glover ed.: 1985); Oligonucleotide Synthesis (Gait ed.: 1984); Nucleic Acid Hybridization (Hames & Higgins eds.: 1985); Transcription And Translation (Hames & Higgins, eds.:1984); Animal Cell Culture (Freshney, ed.:1986); Immobilized Cells And Enzymes (IRL Press:1986); Perbal, A Practical Guide To Molecular Cloning (1984); Ausubel et al., eds. Current Protocols in Molecular Biology, (John Wiley & Sons, Inc.:1994); and Harlow and Lane. Antibodies: A Laboratory Manual (Cold Spring Harbor Laboratory Press: 1988).

The terms “vector”, and “expression vector” mean the vehicle by which a DNA or RNA sequence (e.g. a foreign gene) can be introduced into a host cell, so as to transform the host and promote expression of a polypeptide (e.g. transcription and translation) of the introduced sequence.

### EXAMPLES

### Example 1

### MuSK is Upregulated During Long-Term Memory Consolidation

### Materials and Methods

Inhibitory Avoidance (IA) Training

IA training was carried out as previously described (Milekic and Alberini (2002), Neuron 36: 521-525). Briefly, during training sessions, the rat was placed in the safe compartment and after 10 seconds, the door was opened allowing the rat access to the shock compartment. Here the rat received a foot shock (0.6 mA for 2 seconds) and was then returned to the home cage. Memory was tested by replacing the animal back into the safe compartment and measuring the latency (in seconds) of the rat to reenter the shock chamber. The experimental group consisted of rats that were exposed to the IA apparatus, shocked and tested 20 hours (20 h+) or 24 hours (24 h+) after training. Control groups consisted of: 1) rats exposed to the IA apparatus without receiving the shock and killed immediately after (0 h−), 2) rats that were exposed to the IA apparatus without receiving the shock and killed 20 or 24 hours following exposure to the IA apparatus (20 h−; 24 h−) and 3) rats that were exposed to the IA context 1 hour after receiving the shock (unpaired). After testing, rats were anesthetized and their hippocampi were rapidly dissected and frozen for Western blot.

Microarray Data and TaqMan Real-Time PCR

Total RNA from hippocampi obtained from control (0 h− and 20 h−) and trained (20 h+) groups of rats were hybridized to Affymetrix U34 rat arrays. Quadruplicates of two independent experiments were carried out. The overall gene expression was analyzed according to the Affymetrix algorithm-based change. Genes that significantly changed their expression levels in trained conditions versus controls were identified by using a Student's t test (p<0.05) and genes with >1.8-fold changes were selected.

To validate the microarray data, TaqMan quantitative reverse transcriptase-polymerase chain reactions (RT-PCR) were performed. Two primers and one probe were designed for each gene using PrimerExpress3 software (Applied Biosystems) (Forward primer: 5′-TGCGCCTATGTTGGAGCAA 3′ (SEQ ID NO: 15); Reverse Primer: 5′-CCTCTGCTCTCTCGCACATG 3′ (SEQ ID NO: 16); probe: 5′-FAM-TGCCTGCAGACAGACCCAGC 3′ (SEQ ID NO: 17)).

Western Blot Analysis

Western blot analyses were carried out as previously described (Taubenfeld et al., (2001) J. Neurosci. 21:84-91). Briefly, extracts from different rat tissue were obtained by polytron homogenization in cold lysis buffer with proteases inhibitors (0.2 M NaCl, 0.1 M HEPES, 10% glycerol, 2 mM NaF, 2 mM Na₄P₂O₇, 5 mM EDTA, 1 mM EGTA, 2 mM DTT, 0.5 mM PMSF, proteases inhibitors cocktail, (Sigma)). Twenty-five mg/lane were resolved on 7.5% SDS-PAGE gels and transferred to Hybond-P membranes (Amersham) by electroblotting. Primary antibodies: Abgent cat # AP7664A; (San Diego, Calif.), anti-GAPDH (Chemicon), anti-actin (Chemicon). Quantitative densitometry analysis was performed using NIH image.

### Results

To identify genes that are regulated during memory consolidation, DNA microarray hybridizations were screened for genes differentially expressed in the hippocampus after inhibitory avoidance (IA) learning. Results from hybridizations of Affimetrix rat arrays (U34), containing oligonucleotide sequences that enable the analysis of more than 7,000 full-length genes and 17,000 ESTs, were statistically analyzed using Student's t-test. The concentration of one hundred sixty transcripts significantly changed between trained and control conditions. One transcript that showed a statistical significant increase was MuSK.

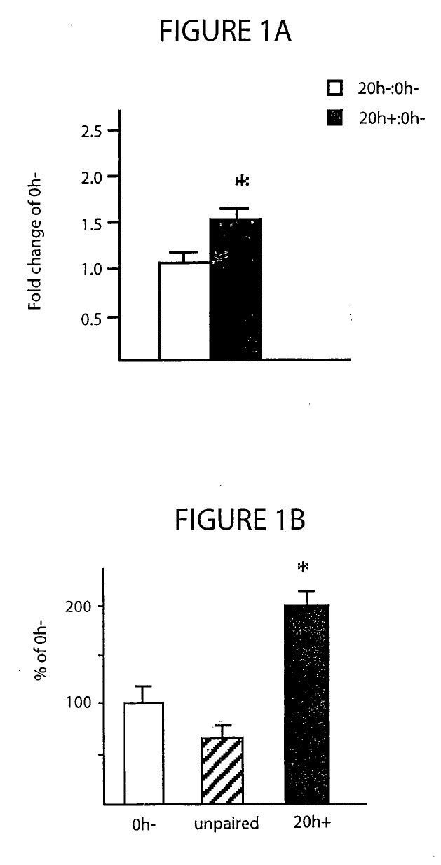

A validation study was then carried out to confirm the array result. Real-time PCR was performed using TaqMan and a sequence-specific intercalator. Amplification plots and melting temperature analysis were generated using the cDNA from hippocampi of trained or control rats as a template. FIG. 1A shows that MuSK mRNA was significantly upregulated (p<0.05) in the hippocampus at 20 hours after training (20 h+) compared to control groups that were either exposed to the IA context without receiving a shock and sacrificed immediately after (0 h−) or 20 hours later (20 h−).

The increase in MuSK mRNA was accompanying by a parallel rise in the levels of a protein recognized by Abgent antibody cat# AP7664A. Quantitated Western blot analyses (FIG. 1B), carried out on hippocampal protein extracts obtained from 0 h−, 20 h unpaired and 20 h+, revealed a significant induction of the protein recognized by Abgent antibody cat # AP7664A in the trained groups compared to controls (0 h−, n=4, 100±28.4%; 20 h unpaired, n=4, 62.3±6.9%; 20 h+, (n=8), 173.26±19.10%). Twenty hours after training the expression levels of this protein was significantly higher than that of the 0h− and unpaired control groups (p<0.05 for both).

### Example 2

### Cloning of MuSK in Rat Brain and Characterization of MuSK Expression in Various Tissues

### Materials and Methods

Cloning of Brain MuSK

Total RNA from various regions of the adult rat brain and hippocampal cell cultures was isolated with TRIzol reagent (Invitrogen; Carlsbad, Calif.) and reverse transcription was performed using oligo (dT) (Invitrogen). Five sets of 20mer primers were designed based on the sequence of MuSK cloned from rat muscle (GenBank accession number U34985) and used for PCR amplifications. Primers: starting from the 5′ end of MuSK sequence, forward (F) 1: 5′-TTACAGATGCTCACCCTGGT (SEQ ID NO: 3); reverse (R) 1: 5′-CTTAATCCAGGACACGGATGG (SEQ ID NO: 4); F2: 5′-CAAGCCATCCGTGTCCTGGAT (SEQ ID NO: 5); R2: 5′-ACAGTAGCCTTTGCTTTCTT (SEQ ID NO: 6); F3: 5′-AGTATAGCAGAATGGAGCAA (SEQ ID NO: 7); R3 5′-GGAAGGCAATGTGGTGAGGGT (SEQ ID NO: 8); F4: 5′-CTGCCGAAGGAGGAGAGAGTG (SEQ ID NO: 9); R4: 5′-GTTTCCATCAGCTTTGTA GTA (SEQ ID NO: 10); F5: 5′-AGGAACATCTACTCCGCAGAC (SEQ ID NO: 11); R5: 5′-TGAAAAGATCCTCCTGGGTG (SEQ ID NO: 12). Five overlapping PCR products of 439 bp, 508 bp, 717 bp, 723 bp and 409 bp that span the entire coding sequence of MuSK were obtained and sequenced.

Western Blot Analysis.

See above.

### Results

As described in Example 1, an analysis of differentially expressed transcripts in the hippocampus after inhibitory avoidance training using Affimetrix microarray hybridizations revealed that MuSK was one of the transcripts significantly increased. The upregulation of MuSK during memory consolidation was confirmed by real-time PCR. Thus, it was further investigated whether MuSK was expressed in the brain, even though it had previously been reported that MuSK was not expressed in the mammalian brain (Smith and Hilgenberg, (2002) NeuroReport 13(12): 1485-95).

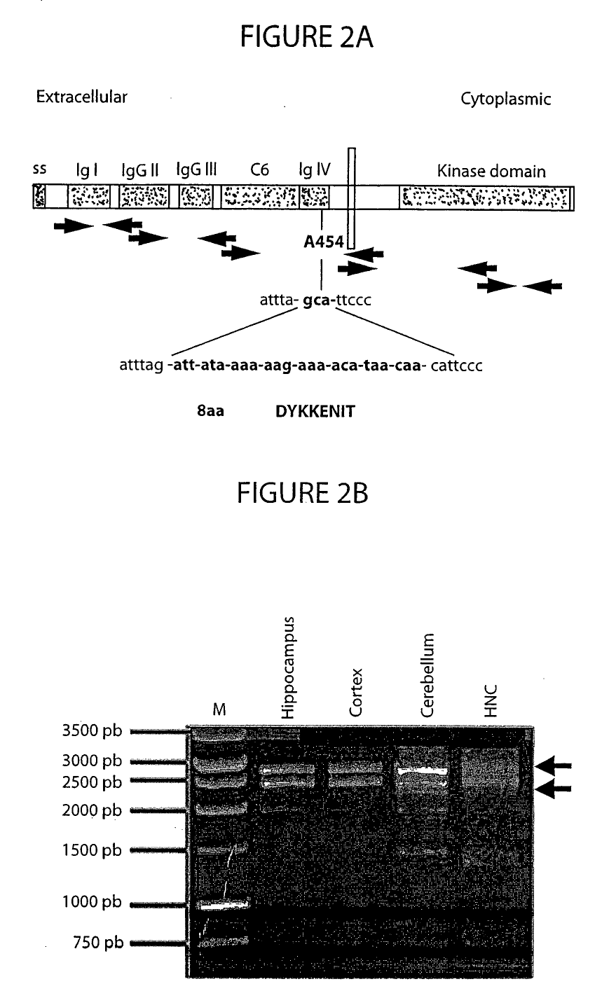

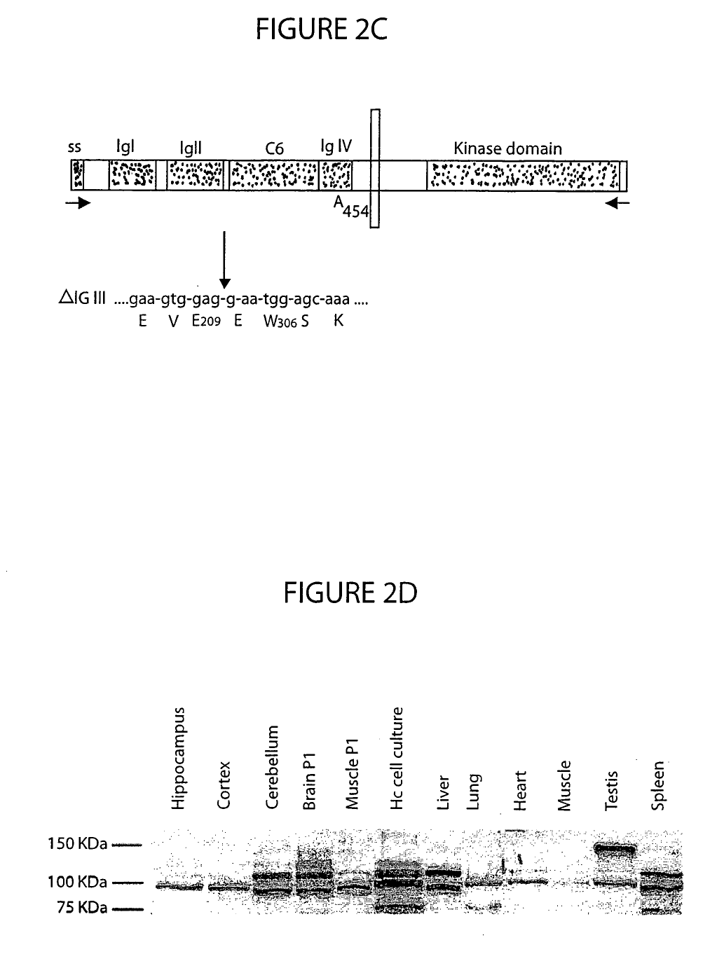

Toward this end, sets of oligodeoxynucleotides primers for PCR fragment amplifications that spanned the entire muscle MuSK cDNA sequence (GenBank accession U34985) were designed. These primers were used for PCR amplifications of cDNAs obtained from hippocampus, cortex, cerebellum and hippocampal cultures (FIG. 2A). In all samples, fragments that had the expected size, according to the muscle MuSK sequence, were amplified. The sequence of all these fragments confirmed that all bands amplified from all brain regions as well as hippocampal culture cDNAs were identical, except for one difference, to the corresponding sequence of muscle MuSK. In all the brain regions and in hippocampal cell cultures, the MuSK sequence contained a deletion of bases 1481-1504 of the muscle MuSK sequence (GenBank accession number U34985), resulting in an in-frame substitute of alanine in lieu of an eight amino acid sequence (aspartate, tyrosine, lysine, lysine, glutamate, asparagine, isoleucine and threonine) found in the muscle MuSK polypeptide (FIG. 2A). Such modification had already been described in mouse skeletal myotubes (Ganju et al., (1995) Oncogene 11: 281-290; Hesser et al., (1999) FEBS Lett. 442: 133-137). The nucleotide and amino acid sequences of one isoform of brain MuSK are depicted in SEQ ID NOS: 1 (and 20) and 2, respectively (SEQ ID NO: 1 contains 5′ and 3′ non-coding sequences; SEQ ID NO: 20 is the coding sequence, without these non-coding sequences; SEQ ID NO: 2 is the amino acid sequence encoded for by both of these sequences).

Two MuSK isoforms are expressed in the brain. Sets of oligodeoxynucleotide (ODN) primers for the amplification of the MuSK full length open reading frame (F: 5′-ATGAGAGAGCTCGTCAACAT-3′ (SEQ ID NO: 21); R: 5′-TGAAAAGATCCTCCTGGGTG-3′ (SEQ ID NO: 22)) were employed in RT-PCR amplifications of cDNAs obtained from hippocampus, cortex, cerebellum and embryonic day 18 (E18) HNC. Gel electrophoresis analysis revealed that two bands were amplified (FIG. 2B). Sequencing of these bands showed that they corresponded to two alternatively spliced MuSK transcripts, which differed by the presence or absence of the third Ig-like domain. One isoform was 2,644 bp long and corresponded to the isoform described in muscle except that it had the A₄₅₄ substitution (substitution of alanine in lieu of an eight amino acid sequence (aspartate, tyrosine, lysine, lysine, glutamate, asparagine, isoleucine and threonine). The second MuSK transcript found in all brain regions and HNC was shorter (2,359 bp) and, compared to the longer muscle MuSK sequence (U34985) carried two distinctive features: the A₄₅₄ substitution (substitution of alanine in lieu of an eight amino acid sequence (aspartate, tyrosine, lysine, lysine, glutamate, asparagine, isoleucine and threonine)) and a deletion of the third Ig-like domain (FIG. 2C). Thus, both MuSK isoforms expressed in the brain have the 8 amino acid-Ala substitution, but differ by the presence or absence of the third Ig-like domain. An isoform lacking the third Ig-like domain, but without the A₄₅₄ substitution, had been previously found in denervated rat muscle by Hesser et al. (1999) and named MuSK-ΔIgIII. Interestingly, this protein has been shown to mediate muscle AChRs clustering similarly to the long isoform (Hesser et al., 1999). A MuSK protein carrying both the A₄₅₄ substitution and ΔIgIII has not previously been described. The amino acid sequence of this MuSK isoform is set forth in SEQ ID NO: 19.

The expression level of the protein recognized by Abgent antibody cat # AP7664A in various tissues including hippocampus, cortex, cerebellum, post-natal day 1 brain (brain p1) and muscle (Muscle p1), primary hippocampal cultures, heart, muscle, liver, testis and spleen was determined. The results of Western blot analyses revealed that this protein is ubiquitously expressed (FIG. 2B). A reference protein for loading comparisons could not be used because most proteins, including housekeeping factors are differentially expressed in different tissues. Thus, the relative concentration is referred to the same concentration of total protein loaded in each sample. Two distinct antisera were used to carry out independent Western blot analyses of the same tissue extracts; all confirmed the presence of this protein in all the tissues analyzed. These results demonstrate that the protein recognized by Abgent antibody cat # AP7664A is ubiquitously expressed.

### Example 3

### MuSK is Required for Memory Formation

### Materials and Methods

Cell Culture and Treatment with Antisense Oligodeoxynucleotides (ODNs)

Cell cultures were prepared from hippocampi of embryonic day 18 Long-Evans rats as described previously (Goslin and Banker, Rat hippocampal neurons in low density culture. In Culturing Nerve Cells, (1991) MIT Press, Cambridge Mass., 251-282; Benson et al., (1994) J Neurocytology 23(5):279-95), with some modifications (described herein). Cells were dissociated by treatment with 0.25% trypsin for 15 min at 37° C. followed by trituration through a Pasteur pipette. Cells were plated at a density of 1.3×10⁴ cells/cm² on poly-L-lysine-coated coverslips in minimum essential media (MEM; Invitrogen; Carlsbad, Calif.) containing 10% horse serum. After ˜3 hr, when cells had attached, coverslips were transferred to dishes containing Neurobasal medium supplemented with B-27 (Invitrogen; Carlsbad, Calif.) where they were maintained for the entire time of culture. Four-day-old hippocampal cultures were treated with 5 μM of either MuSK antisense ODN (MuSK-ODN: 5′-GAATGTTGACGAGCTCTCTCATG-3′; SEQ ID NO: 13), scrambled antisense ODN (Sc-ODN: 5′-TACTATGGATCGTCTGCGCATAG-3′; SEQ ID NO: 14) or vehicle (phosphate buffered saline (PBS)) every day for 3 consecutive days. Both ODNs were phosphorothioated on the three terminal bases of the 5′ end and the three terminal bases of the 3′ end. Both ODNs were reverse phase chromatography-purified and obtained from Gene Link (Hawthorne, N.Y.).

Inhibitory Avoidance Training (IA) Training

See above.

### Results

To further investigate the role of MuSK in memory formation, the expression of MuSK in the hippocampus was knocked down by injecting antisense-ODN and measuring the resulting effect on memory retention (Guzowski et al., (1997) Proc. Natl. Acad. Sci. USA 94: 2693-2698; Guzowski et al., (2000) J. Neurosci. 20: 3993-4001; Taubenfeld et al., (2001) Nature Neurosci. 4:813-818), who showed that the injected ODN diffuses throughout the entire dorsal hippocampus.

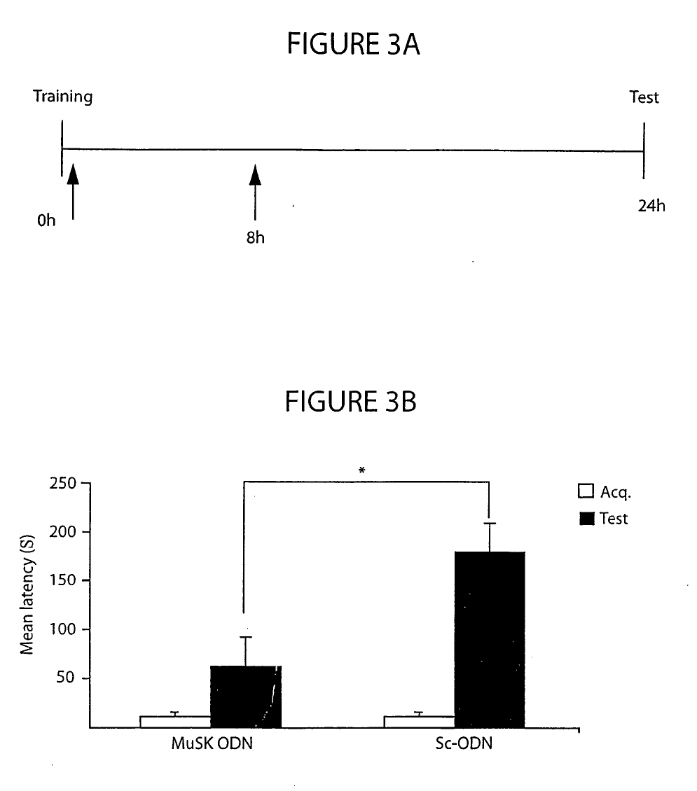

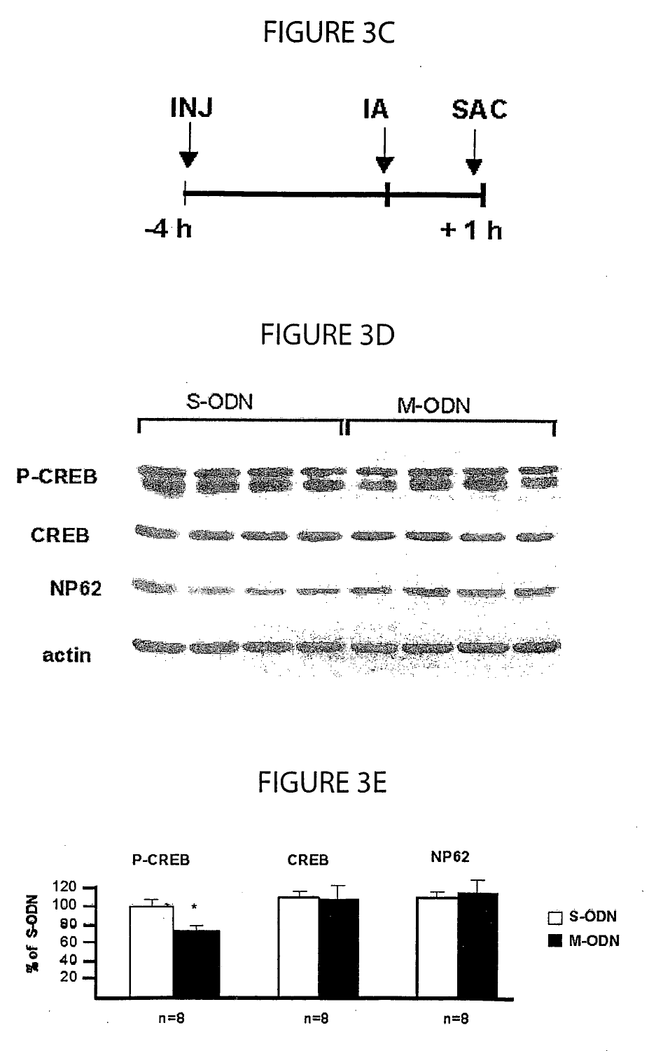

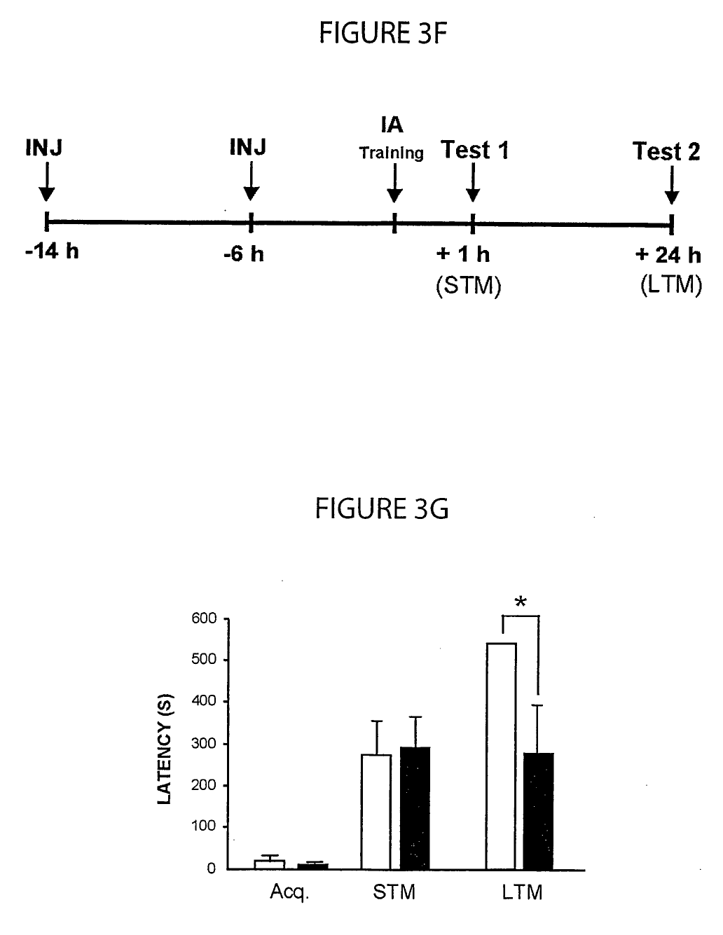

Groups of rats bilaterally implanted with cannulas were trained and injected with either MuSK antisense-ODN, Sc-ODN or PBS immediately after and 8 hours after training. Memory retention was tested 24 hours after training. Antisense treatment produced a significant impairment (p<0.05 Student's t test) in memory retention (61.46±29.3 s) compared to Sc-ODN (180.35±27.6 s) and PBS control groups (FIG. 3B). No significant difference was observed between the Sc-ODN and PBS-injected groups. Therefore, the two were combined for statistical analysis. These results demonstrate that MuSK is required for memory formation.

Experiments were performed to determine that, although required for consolidation of long-term memory, MuSK is not required for memory acquisition or short term memory. Expression of MuSK in rat hippocampi was knocked-down by bilaterally injecting MuSK-ODN 14 and 6 hours before training (FIG. 3F) and determining the effect of this treatment on memory retention 1 hour (short term memory) and 24 hours (long term memory) after training. As shown in FIG. 3G, unlike the post-training MuSK-ODN injections that impaired retention, the pre-training MuSK-ODN or Sc-ODN injections did not affect the acquisition or short term memory in the IA task. In contrast, the long-term memory was partly, but significantly disrupted in the MuSK-ODN (281.1±115.4%) treated group compared to Sc-ODN control group (540 s, n=8, student t test: p<0.05). Thus, MuSK is not required for learning or short-term memory, but is required for the consolidation of long-term memory.

Experiments have also shown that hippocampal knock-down of MuSK blocks both consolidation of inhibitory avoidance (IA) memory and the induction of the transcription factor CCAAT enhancer binding protein β (C/EBP β) following IA training. Additional experiments were conducted to determine whether C/EBP β upstream mechanisms such as the activation of the transcription factor cAMP response element binding protein (CREB) are affected by MuSK disruption. Phosphorylation of CREB on Ser 133 (pCREB) is an essential step for the activation of CREB, and activation of this factor is known to be required for memory formation. IA training causes the induction of C/EBPβ in the same hippocampal neuronal population that several hours earlier showed an activated CREB response (Taubenfeld et al. 2001). However, it is unclear whether the antisense-mediated hippocampal knock-down of MuSK affects pCREB. Since hippocampal pCREB is significantly increased immediately after IA training (Bernabeu, 1997, Taubenfeld et al., 1999, 2001), to ensure that MuSK disruption was achieved at this time, MuSK antisense (MuSK-ODN) or its scrambled control sequence (Sc-ODN) was injected bilaterally into rat hippocampi four hours before training and rats were sacrificed one hour after training (FIG. 3C). Hippocampal protein extracts were assessed for the levels of pCREB using quantitative Western blot analyses (four representative blots are shown in FIG. 3D). As shown in FIG. 3E, hippocampal levels of pCREB were significantly and selectively reduced by MuSK-ODN (71.9±8.1%; n=8), compared to Sc-ODN injection (100%±6.6%; n=8; student t test: p<0.05). To determine whether the pCREB decrease resulted from post-translational modification or change in CREB expression, the same membranes were stained with anti-CREB antibody. As depicted in FIG. 3D, the expression levels of CREB (MuSK-ODN, 99.4±12.1%; Sc-ODN, 100±6.3%) remained unchanged. Moreover, to confirm the selectivity of the MuSK-ODN effect, the same membranes were tested for the expression level of another nuclear protein, nuclear protein 62 (NP62), which, as shown in FIG. 3D, also remained unchanged (MuSK-ODN, 105.2±13.5%; Sc-ODN, 100±4.0%). In each experiment, pCREB, CREB and NP62 levels were normalized against the relative concentration of actin, which was used as loading control. Together, these data suggest that if hippocampal MuSK expression is blocked at the time of IA training and immediately after, the learning-induced activation of CREB is completely disrupted.

### Example 4

### Treatment with Abgent Antibody Cat # AP7664A Produces Memory Enhancement

### Materials and Methods

Inhibitory Avoidance Training (IA) Training

See above. Antibodies: Abgent antibody cat # AP7664A (San Diego, Calif.).

### Results

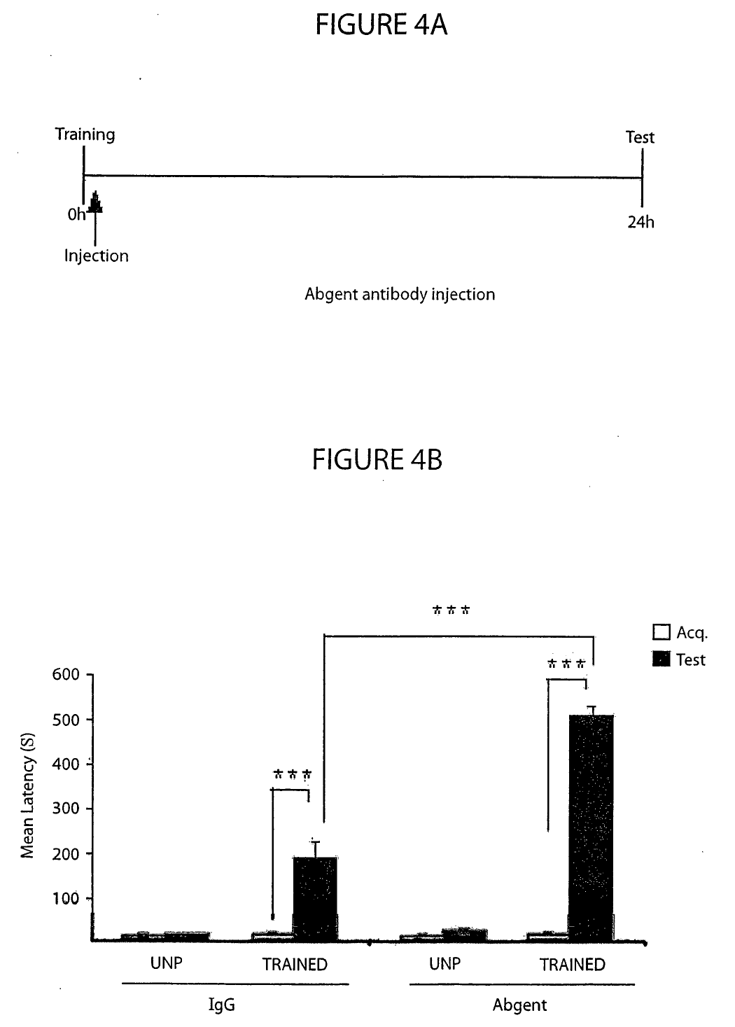

The effect of Abgent antibody cat # AP7664A on IA memory retention was tested next. Rats were trained and, immediately after, received intrahippocampal injections of either Abgent antibody (1 μl of a 250 ng/μl solution), control IgG antiserum or vehicle solution. Memory retention was tested 24 hours after training (see FIG. 4A). As shown in FIG. 4B, trained rats that received the Abgent antibody had a strikingly higher retention than controls (IgG). Specifically, most (6 out of 8) rats treated with Abgent antibody reached the cut-off time of retention (540 sec), suggesting that all had a very strong memory, while the mean latency of the IgG-injected group was 165±58.7 sec and that of vehicle (PBS)-injected group was 236±56.0 sec. Because there was no significant difference between the mean latency values of IgG and that of vehicle-injected group, they were combined for statistical analyses (192.05±35.0 sec). A Neuman-Keul post-hoc revealed that the group injected with Abgent antibody had a significantly higher retention compared to the IgG/PBS-injected group (p<0.001). The mean latencies to enter the shock compartment during training (acquisition) were similar in all groups (unpaired-IgG: 7.94±2.40 s; unpaired-Abgent antibody: 9.27±2.29 s; trained-IgG: 15.0±3.23 s; trained- Abgent antibody: 15.4±4.79 s). The mean retention latencies of all unpaired, control groups, whether they received injections of Abgent antibody (21.40±4.46 s) IgG or PBS, (13.8±5.75) were all similar to the acquisition latencies, demonstrating that no memory was formed and that the treatments per se with either Abgent antibody or IgG did not alter the behavior of the animals.

### Example 5

### MuSK Knock-Down Causes Synapse Morphological Chances

### Materials and Methods

Immunocytochemistry

Hippocampal cell cultures on glass coverslips were fixed for 30 min at room RT with 4% paraformaldehyde in PBS, permeabilized for 5 min at RT with 0.3% Triton X-100 and thereafter blocked for 1 h at RT with 2% BSA in PBS. Incubation with primary antibodies was performed in 0.5% blocking solution overnight at 4° C. After washing, cells were incubated at RT with species-specific fluorescence-conjugated secondary antibodies for 1 h. Species-specific fluorescent secondary antibodies Alexa Fluor-488 and Alexa Fluor-568 (Molecular Probes) were used for all immunostainings. Images were captured with a Leica TCS-SP (UV) at the MSSM-Microscopy Shared Resource Facility.

### Results

To determine whether MuSK is involved in synapse formation, the effect of MuSK knock-down in hippocampal cell cultures was investigated. Sister cultures (culture generated on the same day) of E17 rat hippocampal neurons were grown on coverslips and at 4 days of age, when they start to grow synapses, they were treated with 5 μM MuSK antisense (ODNs) or scrambled control oligodeoxynucleotides (Sc-ODNs) for three days. Additional control cultures remained untreated.

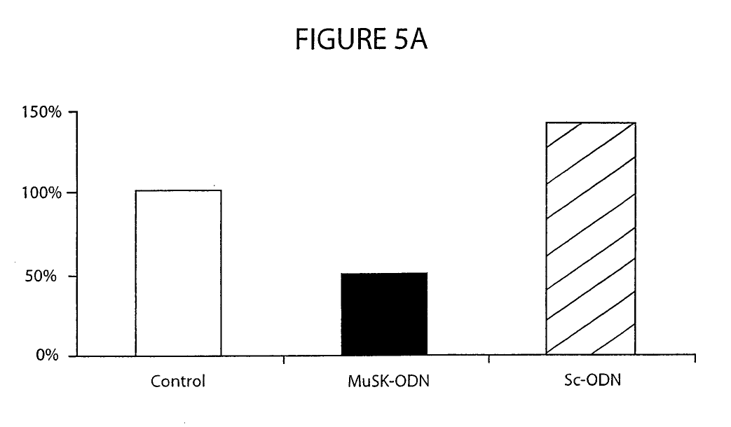

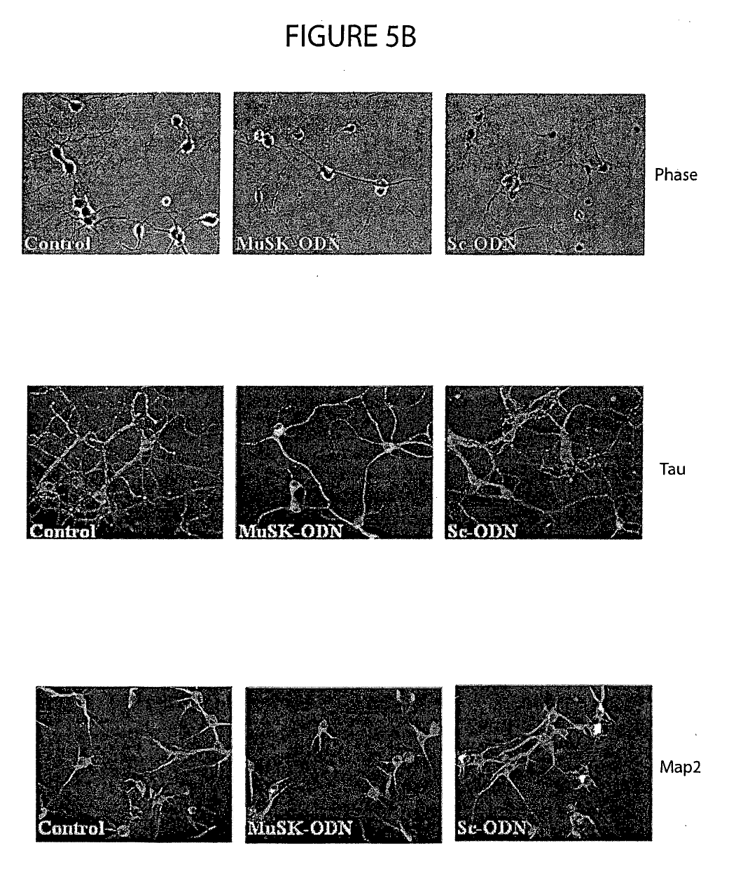

The effect of MuSK antisense on the expression of the protein recognized by Abgent antibody catalog # AP7664A (San Diego, Calif.) was evaluated with quantitated Western blot analyses of cultures treated for 3 days. As depicted in FIG. 5A, MuSK antisense-ODN produced a significant decrease in the expression of the protein recognized by Abgent antibody catalog # AP7664A compared to Sc-ODN treatment and untreated controls.

On the day after the end of the three-day treatment, the morphology of the cultures was analyzed by double-staining them with the dendritic and axonal markers anti-MAP2 and anti-Tau, respectively (see FIG. 5B). The morphology of the antisense-treated cultures was profoundly different than the control and scrambled-ODN treated neurons. The knock-down of MuSK in hippocampal cultured neurons caused profound morphological changes. The neuronal processes of the antisense-treated cells (MuSK-ODN) are much less developed compared to scrambled-treated (Sc-ODN) and non-treated controls. Moreover, in the antisense-treated cells it was evident that the axonal processes are fewer and more elongated than in both scrambled-treated and non-treated controls.

### Example 6

### Abgent Antibody Recognizes a Protein that Colocalizes with Agrin and Nicotinic Acetylcholine Receptor

### Materials and Methods

Immunohistochemistry

Immunohistochemistry was carried out as described in Taubenfeld et al., ((2001) J. Neurosci. 21:84-91). Briefly, animals were perfused transcardially with cold 4% paraformaldehyde in PBS. Brains were post-fixed overnight in the same fixative with 30% sucrose and then cryoprotected overnight in 30% sucrose and PBS. Twenty-micrometer sections were cut in the coronal plane on a freezing microtome. Immunostaining was performed on free-floating slices. After a series of preincubations in 0.3% Triton X-100 and 10% BSA for 30 min each, sections were incubated with primary antibodies for 48 hr at 4° C., washed three times with PBS, and then treated with a fluorescence-conjugated secondary antibodies in 2% BSA for 30 min at RT. Slices were finally washed three times in PBS, mounted on gelatin-coated slides using Vectashield Mounting Medium (Vector Laboratories, Burlingame Calif.), air-dried and coverslipped. Confocal laser scanning microscopy was performed on Leica TCS-SP (UV) at the MSSM-Microscopy Shared Resource Facility.

Immunocytochemistry

See above.

Antibodies

Primary antibodies: Rabbit anti-MuSK antiserum, # 83033, kindly provided by Dr. Steve Burden (Skirball, NYU), mouse anti-agrin (Chemicon, Temecula, Calif.), mouse anti-AChRα7 (Covance Research Products, Berkeley, Calif.), mouse anti-GluR1 (Santa Cruz Biotechnology, Santa Cruz, Calif.), mouse anti-GABA_{A}R β-2,3 subunit (Chemicon), mouse anti-Map2 (Sigma, St. Louis, Mo.), mouse anti-Tau (Chemicon). Fluorescence-conjugated α-bungarotoxin (α-Btx-488) (Molecular Probes, Eugene, Oreg.). Species-specific fluorescent secondary antibodies Alexa Fluor-488 and Alexa Fluor-568 (Molecular Probes) were used for all immunostainings.

### Results

In muscle, MuSK interacts with agrin, leading to clustering of acetylcholine receptors (AChRs). In order to investigate whether in the brain MuSK interacts with the same proteins and plays the same functional roles as it does in muscle, the expression pattern of MuSK in the brain in relationship to that of agrin, AChRs, and the primary excitatory and inhibitory receptors for glutamate and GABA was determined. Additionally, the expression pattern of MuSK was explored in relation to these neuronal markers. MuSK expression was also studied in relation to the following synaptic, dendritic and axonal markers: post-synaptic density protein 93 (PSD93) and 95 (PSD95), synapsin, microtubule associated protein 2 (MAP2) and TAU.

Double stainings in rat adult brain sections carried out using Abgent antibody and the following: agrin, AchRα7, GluR1 or GABA_{A}β subunit showed staining in several brain regions, including hippocampus, cortex and cerebellum. The hippocampal staining revealed that neurons of the polymorphic layer and granule cells of the dentate gyrus express the protein recognized by the Abgent antibody. In the CA1, CA2 and CA3 regions of the hippocampus, this protein was mostly localized to the cytoplasm and to the proximal dendrites of pyramidal neurons. In the cerebral cortex, immunoreactivity to this protein was widespread in several neuronal populations of the cortical layers with a strong labeling in layer V pyramidal neurons. Like in the hippocampus, cell bodies and proximal dendrites showed the strongest positivity. In the cerebellum, immunolabelling with Abgent antibody was strongest in the Purkinje cells, while granule cells were mostly negative.

Notably, Abgent antibody staining showed a striking high rate of co-localization with both agrin and AChR staining in all regions, whereas double stainings with GluR1 and GABA_{A} showed a lower degree of overlapping. GABA_{A} receptor immunostaining was seen mostly in the processes whereas Abgent antibody reactivity is most evident in the cell bodies. Representative examples of double-labeled puncta were evident in the pyramidal cells of layer V of cerebral cortex. A relatively high degree of double staining was observed in granule cells of the cerebellum.

To explore at the single cell level the expression and co-localization of MuSK, using rabbit anti-MuSK antiserum, # 83033, AChRs, GluR1 and GABA_{A}, double stainings of rat hippocampal primary cultures were performed. E17 hippocampal neurons were grown on coverslips (Goslin and Banker, Rat hippocampal neurons in low density culture. In Culturing Nerve Cells, (1991) MIT Press, Cambridge Mass., 251-282; Benson et al., (1994) J Neurocytology 23(5):279-95) and stained at day 10-12 of age. Virtually all neurons appeared positive for MuSK. Immunostaining of primary hippocampal neurons with antibodies specific for MuSK revealed positive reactivity distributed throughout the cell bodies and processes. The staining was more diffused in the cell bodies and was distributed in puncta on processes.

The double stainings revealed that, in hippocampal cultures, like in the adult brain, agrin and AChRs often colocalized with MuSK. MuSK and α-Bungarotoxin (a marker of AChR receptors) (e-h) showed abundant points of colocalization. Moreover, double stainings of MuSK/GluR1 and MuSK/GABA_{A} receptors showed that all neurons were also positive for both receptors. However, the pattern of intracellular colocalization appeared to be less widespread than that observed with AChR and agrin.

To examine in further detail the membrane and pre- or post-synaptic localization of MuSK, a series of double immunostainings was performed in HNC using the rabbit anti-MuSK antiserum, # 83033 and antibodies specific for the following synaptic, dendritic and axonal markers: post-synaptic density protein 93 (PSD93) and 95 (PSD95), synapsin, microtubule associated protein 2 (MAP2) and TAU. While PSD-95 is a critical component of the post-synaptic densities (PSD) at glutamatergic synapses (Sheng, 2001), PSD-93 plays a similar role at the neuronal nicotinic cholinergic synapses (Conroy et al., 2003; Parker et al., 2004). Synapsin, on the other hand, as a synaptic vesicle associated protein, represents a general marker of all synapses at their pre-synaptic sites (De Camilli and Greengard, 1986). MAP2 and TAU are generally used to differentiate between dendritic and axonal compartments of neurons, respectively (Caceres et al., 1984, De Camilli et al., 1984, Binder et al., 1985).

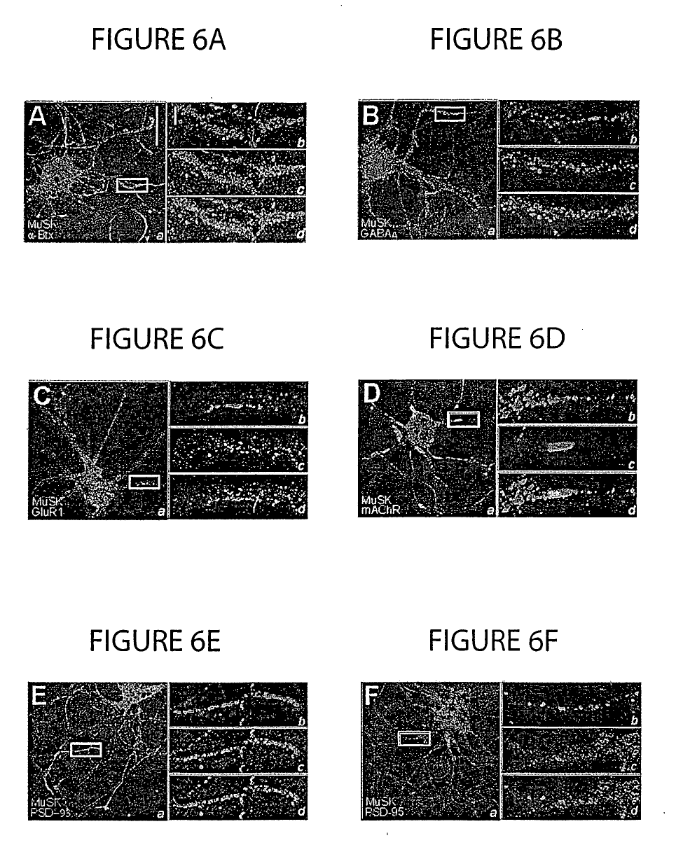

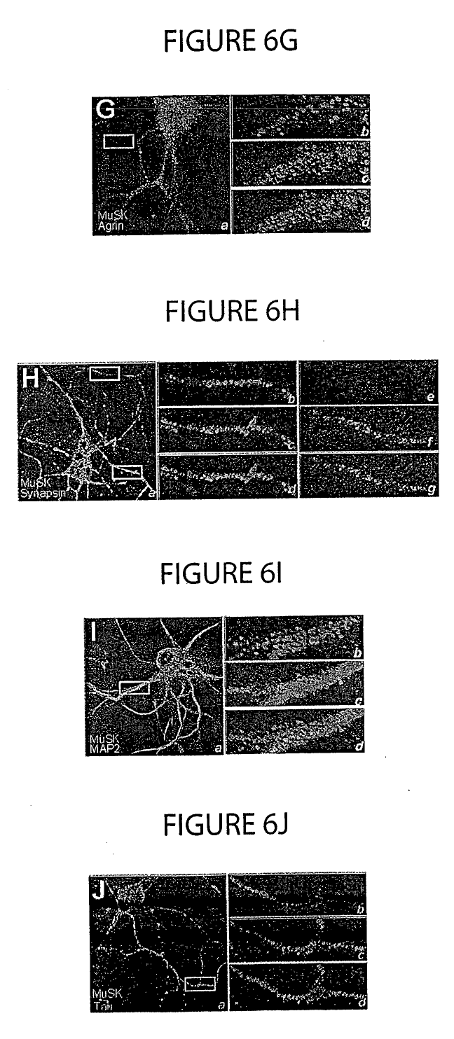

Similar to intracellular MuSK, membrane expression of MuSK largely co-localized with that of nicotinic acetylcholine receptors (nAChRs), as revealed by α-bungarotoxin stainings, but not with that of GABA_{A} and GluR1 receptors (compare FIG. 6A to 6B and 6C) and only partially co-localized with muscarinic acetylcholine receptor (AChR) (FIG. 6D). Notably, these results were further confirmed by the finding that MuSK highly co-localized with PSD93 but not with PSD95 (FIGS. 6E and F). These data strongly suggest that MuSK is likely to interact with neuronal nAChRs but not with glutamate or GABA receptors.

On the other hand, double staining of membrane-expressed MuSK and agrin revealed that the degree of co-expression of these proteins in HNC is marginal compared to that observed in brain sections (FIG. 6G). A possible explanation for this discrepancy is that these two proteins might be synthesized in the same cells. Therefore, they would appear to largely co-localize in permeabilized brain sections,—but not necessarily in membrane double staining,—. In agreement with this hypothesis, similar to brain section stainings, MuSK/agrin double staining of permeabilized HNC showed a large degree of intracellular co-localization.

Double staining of MuSK/synapsin (FIG. 6H) revealed that virtually all MuSK-positive processes were also synapsin-positive. Analysis at high magnification showed that along the double positive processes, the MuSK positive puncta partially overlapped or appeared to be adjacent to synapsin-positive puncta. Similar results were obtained with double staining of MuSK with the other vesicle pre-synaptic marker synaptophysin. Moreover, it was clear that several processes expressed synapsin without MuSK (e.g. FIG. 6H, panels e-g). Thus, we concluded that, as expected, MuSK was expressed at some, but not all synapses. Moreover, in light of the results obtained with the MuSK/PSD93 double staining we hypothesize that MuSK is mostly expressed post-synaptically. This hypothesis was supported by the outcome of the double stainings MuSK/MAP2 and MuSK/TAU. While most processes showed overlapping MuSK/MAP2 stainings, suggesting that MuSK is generally expressed in dendritic compartments, MuSK and TAU overlapped only partially in some branches (FIGS. 6I and J). However, this partial MuSK/TAU overlapping may suggest that MuSK is expressed also pre-synaptically, at least in some neuronal populations.

### Example 7

### Agrin is Upregulated During Long-Term Memory Consolidation and Increases MuSK Expression

### Materials and Methods

Inhibitory Avoidance (IA) Training

See above.

Western Blot

Anti-agrin antibodies: AGR-520 (Stressgen).

Northern Blot Analysis

Northern blot analyses were performed as previously described (Taubenfeld et al., (2001) J. Neurosci. 21:84-91), with some modification. Total RNA was isolated with TRIZOL Reagent (Invitrogen) according to the manufacturer's protocol. 20 mg of total RNA were electrophoresed on 1.2% agarose gels, transferred to Hybond-N+ membranes and UV-cross linked. The membranes were hybridized overnight at 42° C. with specific probes. The agrin probe was a 1.4 kb fragment that represents a common region to the agrin isoforms. Probes were labeled with random oligonucleotides primers (Prime-It II kit, Stratagene) and (α-P-32) dCTP (Amersham). Quantitative densitometry analysis was performed using NIH image.

### Results

To determine if there was a commensurate upregulation of agrin with the upregulation of MuSK, the levels of agrin during long-term memory consolidation were tested.

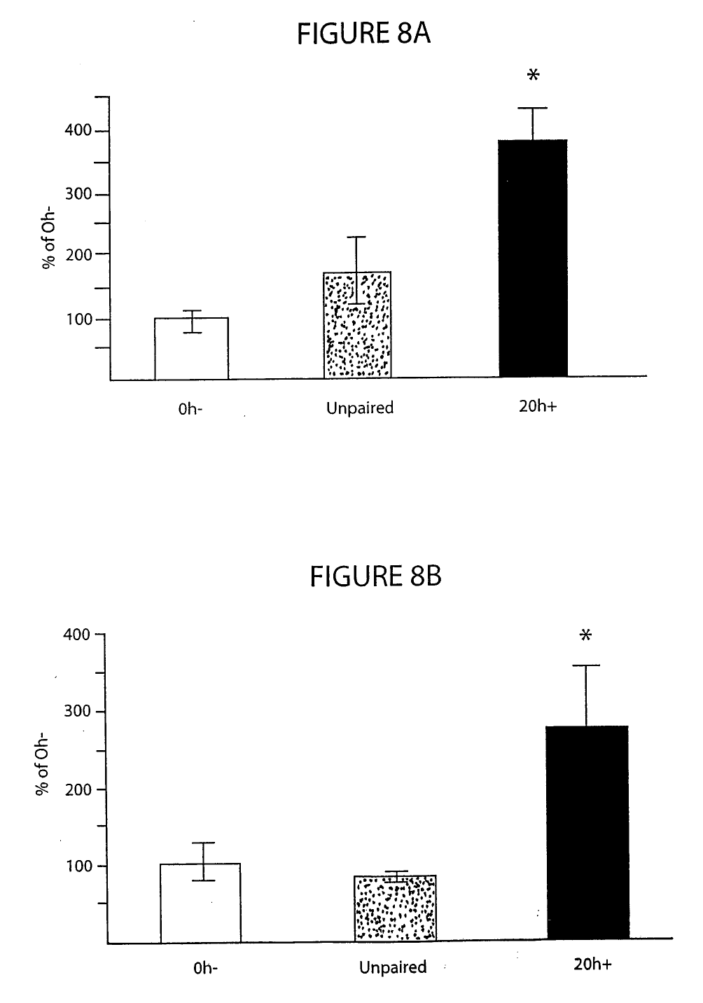

Northern (FIG. 8A) and Western (FIG. 8B) blot analyses of hippocampal extracts taken from control (0 h−, n=4), unpaired (n=4) and IA trained rats (20 h+, n=7) demonstrated that agrin mRNA and protein levels were greater after IA training. Values were normalized to cyclophilin (FIG. 8A) or GAPDH (FIG. 78B). Data are expressed as mean percentage±SEM of the 0 h− (100%) control mean values. Statistical analysis was performed using one-way ANOVA followed by Student-Newman-Keuls test. Trained animals showed a significant increase of agrin mRNA and protein levels compared to 0 h− (*, p<0.05) and unpaired (p<0.05). No significant changes in agrin were found in the unpaired compared with the 0 h− group.

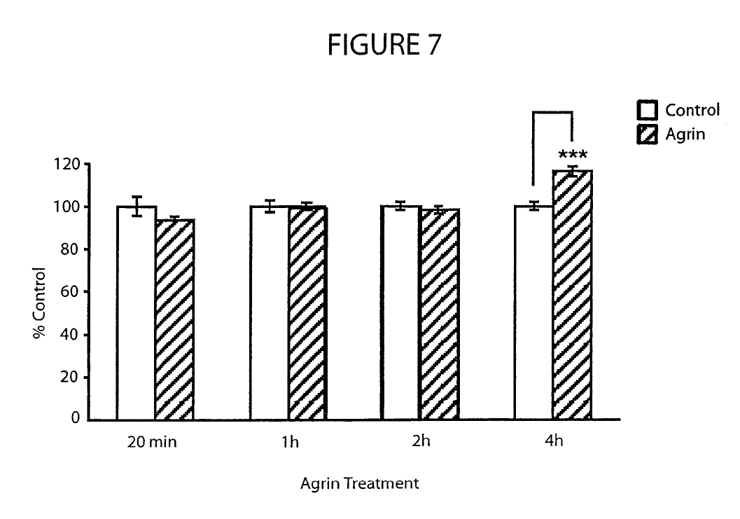

To determine the effect of agrin on the membrane expression of MuSK, HNCs were treated with 5 nM agrin for various amounts of time, including 20 min, 1 h, 2 h and 4 h. Sister cultures were treated with vehicle and were used as controls. At the end of the treatment, cells were fixed and stained with rabbit anti-MuSK antiserum, # 83033. Quantitative morphometric analysis of the membrane expression of MuSK was performed on ten independent fields using IP Lab software. Analysis determined that agrin treatment did not have an effect on membrane expression of MuSK at early time points (20 min, 93±2%, n=10; 1 h, 100±2%, n=10; 2 h, 98±2%, n=10) but significantly increased membrane expression of MuSK at 4 h (116±2%, n=10; Student t test: p<0.001) compared to control treatment (100±2%, n=10) (FIG. 7). These data suggest that in brain, as in muscle, MuSK and agrin are parts of the same signaling pathway.

### Example 9

### Abgent Antibody Cat # AP7664A Enhances Recovery from Stroke

### Materials and Methods

Subjects

Ten adult male Long-Evans hooded rats (350-420 g) were group housed (4 animals/cage) in standard laboratory cages on a 12:12 hour light dark cycle throughout the experiment within the Canadian Centre for Behavioural Neuroscience vivarium.

Reach Training

Over the course of several days, all animals were placed on a restricted diet until they reached 90% of their original body weight. A brief period of pre-training was then given to familiarize the rats with the reaching task. This involved placing them into test cages (10×18×10 cm) with floors constructed of 2 mm bars, 9 mm apart edge to edge. A 4 cm wide and 5 cm deep tray filled with food pellets (45 mg; Bioserv) was mounted on the front of the cage. The rats were required to reach outside the cage and retrieve pellets from the tray. All rats remained in pre-training until they had successfully retrieved 10 pellets (approximately 1 hour/day for 2 days). After pre-training, the rats were placed into a Plexiglas cage (11 cm×40 cm×40 cm) with a 1 cm slot located at the front of the cage. Animals were trained for 20 minutes each day to reach through the slot and retrieve food pellets from a table outside the cage (Whishaw and Pellis, Behavioral Brain Research (1990) 41: 49-59). Rats were permitted to use either limb and the preferred limb was noted for each animal. Each session was videotaped and later used to assess reaching performance. A successful reach was scored when the animal grasped the food pellet, brought it into the cage and to its mouth without dropping the pellet. The percentage of successful reaches [(# successful retrievals/the total # of reaches)×100] was then calculated. Animals were trained for approximately two weeks on this task to establish a baseline measure of motor performance (Kleim et al., (2003) Neurological Research, 25: 789-793).

Infarction

Focal ischemic infarcts were created within caudal forelimb area via bipolar electrocoagulation of the surface vasculature (Nudo and Milliken, (1996) J Neurophysiol. 75(5):2144-9; Kleim et al., (2003) Neurological Research, 25: 789-793). The infarct targeted primarily the distal forelimb representations but in some cases included small regions of proximal representations. The coagulated vessels included fine arterial and venous capillaries as well as larger vessels but specifically avoided any bypassing arteries supplying other cortical areas. Coagulation was continued until all vessels within the targeted area were no longer visible and the tissue appeared white.

Post-Infarction Motor Assessment

Three days after the infarction, the animals were again tested daily in the single pellet reaching task (see above). Each session was videotaped and reaching accuracy later scored as the percentage of successful reaches.

### Results

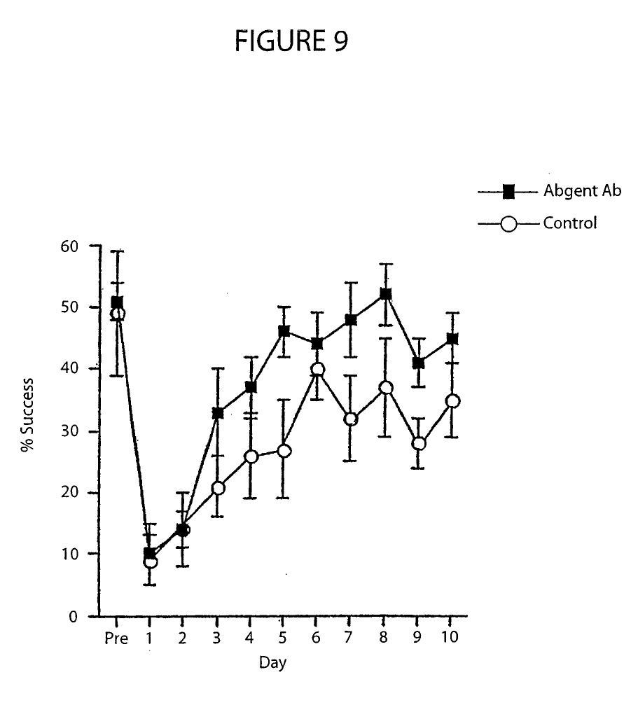

FIG. 9 is a graph demonstrating that delivery of Abgent antibody into the cerebral cortex after focal ischemia enhances motor recovery. Adult male Long-Evans rats were given focal infarctions within forelimb motor cortex. Half of the animals then received MuSK-activating antibody (n=5) or vehicle (n=5) into the damaged cortex.

Skilled reaching ability on the single pellet reaching task was monitored daily after injury (Kleim et al., (2003) Neurological Research, 25: 789-793). A one-way repeated measures ANOVA showed a significant Time x Treatment interaction (p<0.05) where Abgent antibody-injected animals exhibited significantly greater reaching accuracies as training progressed (*Fishers PLSD; p<0.05).

These results suggest that Abgent antibody enhances the recovery of memories after neurological damage, such as stroke. These data also suggest that Abgent antibody might increase the rate at which new memories are acquired.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A method of maintaining or enhancing memory or learning in a mammal comprising activating muscle-specific kinase (MuSK) in the brain of said mammal.

2. The method of claim 1 wherein the section of the brain in which MuSK is activated is the hippocampus.

3. The method of claim 1 wherein the type of memory enhanced is long-term memory.

4. The method of claim 1 wherein the type of memory enhanced is working memory.

5. The method of claim 1 wherein the type of memory enhanced is memory consolidation.

6. The method of claim 1 wherein the mammal is a human.

7. The method of claim 1, wherein MuSK is activated by administering a MuSK-activating agent to said mammal.

8. The method of claim 7, wherein the MuSK-activating agent is a MuSK-activating antibody.

9. A method of maintaining or enhancing memory or learning in a mammal comprising increasing muscle-specific kinase (MuSK) expression in the brain of said mammal.

10. The method of claims 1 or 9 wherein MuSK is a polypeptide comprising the amino acid sequence SEQ ID NO: 2 or SEQ ID NO: 19.

11. The method of claim 10 wherein the MuSK polypeptide is encoded by the nucleotide sequences SEQ ID NO: 1 or SEQ ID NO: 18.

12. The method of claim 9 wherein the increase in MuSK expression is achieved by stabilizing or preventing the degradation of MuSK polypeptide or mRNA.

13. A method of treating a disease or condition associated with memory loss comprising administering an effective amount of a muscle-specific kinase (MuSK)-activating agent to a subject in need of such treatment.

14. A method of treating a neurological impairment comprising administering an effective amount of a muscle-specific kinase (MuSK)-activating agent to a subject in need of such treatment.

15. The method of claims 13 or 14 wherein the MuSK-activating agent is selected from the group consisting of a MuSK-activating antibody, an inhibitor of a MuSK inhibitor, and a MuSK-activating small molecule.

16. The method of claim 15 wherein the MuSK-activating agent is a MuSK-activating antibody.

17. The method of claim 13 wherein the disease or condition associated with memory loss is selected from the group consisting of Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, Parkinson's disease, and stroke.

18. The method of claim 14 wherein the neurological impairment is selected from the group consisting of spinal cord injury, brain trauma, ischemia, shock, head trauma, neuronal injury, neuronal toxicity, neuronal degeneration and stroke.

19. The method of claim 17 wherein the disease is Alzheimer's disease.

20. The method of claim 19 wherein the Alzheimer's disease is in its early stage.

21. The method of claims 17 or 18 wherein the condition is stroke.

22. The method of claims 13 or 14 wherein the subject is a mammal.

23. The method of claim 22 wherein the subject is a human.

24. A method of treating stroke comprising administering an effective amount of a muscle-specific kinase (MuSK)-activating agent to a subject in need of such treatment.

25. A method of identifying compounds that maintain or enhance memory or learning or enhance the recovery from a neurological impairment comprising screening for compounds that increase muscle-specific kinase (MuSK) expression or stability in the brain, wherein an increase in MuSK in the presence of the compound compared to a control in which the compound was not present, indicates that the compound increases MuSK expression or stability.

26. The method of claim 25 wherein the increase in MuSK stability or expression is detected by the method selected from the group consisting of RT-PCR, Northern blot, immunohistochemistry, immunocytochemistry, RNase protection assay, immunoprecipitation, in situ hybridization, or Western blot analysis.

27. The method of claim 25 wherein the screening for compounds that increase MuSK expression or stability is done in whole animals in vivo.

28. The method of claim 25 wherein the screening for compounds that increase MuSK expression or stability is done in ex vivo explants of brain tissue.

29. The method of claim 25 wherein the screening for compounds that increase MuSK expression or stability is done in cultured neurons.

30. A method of identifying compounds that enhance the recovery from a neurological impairment comprising screening for compounds that activate muscle-specific kinase (MuSK) in the brain, wherein an increase in MuSK activity in the presence of the compound compared to a control in which the compound was not present, indicates that the compound increases MuSK activation.

31. The method of claim 30 wherein the increase in MuSK activity is measured by detecting an increase in the phosphorylation of MuSK.

32. The method of claim 30 wherein the increase in MuSK activity is measured by detecting an increase in agrin-MuSK binding.

33. The method of claim 30 wherein the increase in MuSK activity is measured by detecting an increase in acetylcholinesterase receptor phosphorylation.

34. An isolated nucleic acid comprising a nucleotide sequence that is at least 85% identical to SEQ ID NO: 1.

35. An isolated polypeptide encoded for by the nucleic acid of claim 34.

36. A method of treating a disease or condition associated with memory loss comprising administering an effective amount of Abgent antibody catalog # AP7664A to a subject in need of such treatment.

37. A method of treating a neurological impairment comprising administering an effective amount of Abgent antibody catalog # AP7664A to a subject in need of such treatment.

38. The method of claim 36 wherein the disease or condition associated with memory loss is selected from the group consisting of Alzheimer's disease, senile dementia of the Alzheimer's type, senile dementia, brain trauma, age-associated memory impairment, amnesia, ischemia, shock, head trauma, neuronal injury, neuronal toxicity, neuronal degeneration, Parkinson's disease, and stroke.

39. The method of claim 37 wherein the neurological impairment is selected from the group consisting of spinal cord injury, brain trauma, ischemia, shock, head trauma, neuronal injury, neuronal toxicity, neuronal degeneration and stroke.

40. The method of claim 38 wherein the disease is Alzheimer's disease.

41. The method of claim 40 wherein the Alzheimer's disease is in its early stage.

42. The method of claims 38 or 39 wherein the condition is stroke.

43. The method of claims 36 or 37 wherein the subject is a mammal.

44. The method of claim 43 wherein the subject is a human.

45. A method of treating stroke comprising administering an effective amount of Abgent antibody catalog # AP7664A to a subject in need of such treatment.

46. An isolated nucleic acid comprising the nucleotide sequence set forth in SEQ ID NO: 18.

47. An isolated polypeptide encoded for by the nucleic acid of claim 46.

48. A method of treating a disease or condition associated with memory loss comprising administering an effective amount of a compound identified by the method set forth in any of claims 25-33.


SEQUENCE LISTING (22 sequences)

SEQ ID NO: 1 - DNA, 2831 bp, Rattus norvegicus
  misc_feature (1)..(2831): n is a, g, c, or t/u, unknown or other
caaacagtcattagtggacgactctattgtaataaactgtgctttaaaatgtaaaccagggagcgttttttttcctcacattgtccagaagcaacctttcttcctgagcctggattaatcatgagagagctcgtcaacattccactgttacagatgctcaccctggttgccttcagcgggaccgagaaacttccaaaagcccctgtcatcaccacgcctcttgaaactgtagatgccttagttgaagaagtggcgactttcatgtgcgccgtggaatcctaccctcagcctgaaatttcttggaccagaaataaaattctcatcaagctgtttgacacccgctacagcatccgagagaacggtcagctcctcaccatcctgagtgtggaggacagtgatgatggcatctactgctgcacagccaacaatggagtgggaggagcggtggaaagttgtggcgccctgcaagtgaagatgaagcctaaaataactcgtcctcccatcaatgtaaaaataattgagggattgaaagcagtcctaccgtgcactacgatgggtaaccccaagccatccgtgtcctggattaagggggacagtgctctcagggaaaattccaggattgcagttcttgaatctgggagtttaaggatccataatgtgcaaaaggaagacgcaggacagtaccgatgtgtggcaaaaaacagcctgggcacagcttactccaaactggtgaagctggaagtggaggtttttgcaagaatcctgcgtgctcctgaatcccacaatgtcacctttggttcctttgtaaccctacgctgcacagcaataggcatgcctgtccccaccatcagctggattgaaaacggaaatgctgtttcttcaggttccattcaagagaatgtgaaagaccgagtgattgactcaagactccagctctttatcacaaagccaggactctacacatgcatagctaccaataagcatggagagaaattcagtaccgcaaaggctgcagccactgtcagtatagcagaatggagcaaatcacagaaagaaagcaaaggctactgtgcccagtacagaggggaggtgtgtgatgccgtcctggtgaaagactctcttgtcttcttcaacacctcctatcccgaccctgaggaggcccaagagctgctgatccacactgcgtggaatgaactcaaggctgtgagcccactctgccgaccagctgccgaggctctgctgtgtaatcacctcttccaggagtgcagccctggagtgctacctactcctatgcccatttgcagagagtactgcttggcagtaaaggagctcttctgtgcaaaggaatggctggcaatggaagggaagacccaccgcggactctacagatccgggatgcatttcctcccggtcccggagtgcagcaagcttcccagcatgcaccaggaccccacagcctgcacaagactgccgtatttagcattcccgtccataacgtcctccaagccgagcgtggacattccaaacctgcctgcctccacgtcttccttcgccgtctcgcctgcgtactccatgactgtcatcatctccatcatgtcctgctttgcggtgtttgctctcctcaccatcactactctctattgctgccgaaggaggagagagtggaaaaataagaaaagagagtcggcagcggtgaccctcaccacattgccttccgagctcctgctggacaggctgcatcccaaccccatgtaccagaggatgccactccttctgaatcccaagttgctcagcctggagtatccgaggaataacatcgagtatgtcagagacatcggagagggagcgtttggaagggtctttcaagcgagggccccaggcttgcttccttatgaacccttcactatggtggctgtgaagatgctgaaggaggaggcctccgcagatatgcaggcagactttcagagggaggcagccctcatggcggagtttgacaaccccaacattgtgaagctcttaggtgtgtgtgctgttgggaagccaatgtgcctgctctttgaatatatggcctatggtgacctcaatgagttcctccgaagcatgtcccctcacactgtgtgcagcctcagccacagtgacctgtccacgagggctcgggtgtccagccctggtcctccacccctgtcttgtgcggaacagctctgtattgccaggcaagtggcagctggcatggcctacctgtcggagcgcaagtttgtccatcgggacttagctaccaggaactgcctggttggagagaacatggtggtgaaaattgcagactttggcctctctaggaacatctactccgcagactactacaaagctgatggaaacgatgctatacctatccgctggatgccacccgagtctatcttctacaaccgctacaccacggagtcagatgtgtgggcttatggcgtggtcctctgggagatcttctcctatggactgcagccctactatggaatggcccatgaggaggtcatttactatgtgagagatggtaacatccttgcctgccctgagaactgtcccttggaactgtacaaccttatgcgcctatgttggagcaagctgcctgcagacagacccagcttctgcagtatccaccggatcctgcagcgcatgtgcgagagagcagagggaacggtaggcgtctaaggttgaccatgctcaaacaacacccaggaggatcttttcagactgcgagctggagggatcctaaagcagagggcgnataagnncagataggaagagtttatctcaggcagcacgtncagttggttgtt

SEQ ID NO: 2 - protein, 860 aa, Rattus norvegicus
MRELVNIPLLQMLTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSALRENSRIAVLESGSLRIHNVQKEDAGQYRCVAKNSLGTAYSKLVKLEVEVFARILRAPESHNVTFGSFVTLRCTAIGMPVPTISWIENGNAVSSGSIQENVKDRVIDSRLQLFITKPGLYTCIATNKHGEKFSTAKAAATVSIAEWSKSQKESKGYCAQYRGEVCDAVLVKDSLVFFNTSYPDPEEAQELLIHTAWNELKAVSPLCRPAAEALLCNHLFQECSPGVLPTPMPICREYCLAVKELFCAKEWLAMEGKTHRGLYRSGMHFLPVPECSKLPSMHQDPTACTRLPYLAFPSITSSKPSVDIPNLPASTSSFAVSPAYSMTVIISIMSCFAVFALLTITTLYCCRRRREWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSTRARVSSPGPPPLSCAEQLCIARQVAAGMAYLSERKFVHRDLATRNCLVGENMVVKIADFGLSRNIYSADYYKADGNDAIPIRWMPPESIFYNRYTTESDVWAYGVVLWEIFSYGLQPYYGMAHEEVIYYVRDGNILACPENCPLELYNLMRLCWSKLPADRPSFCSIHRILQRMCERAEGTVGV

SEQ ID NO: 3 - DNA, 20 bp, artificial
  primer
ttacagatgctcaccctggt

SEQ ID NO: 4 - DNA, 21 bp, artificial
  primer
cttaatccaggacacggatgg

SEQ ID NO: 5 - DNA, 21 bp, artificial
  primer
caagccatccgtgtcctggat

SEQ ID NO: 6 - DNA, 20 bp, artificial
  primer
acagtagcctttgctttctt

SEQ ID NO: 7 - DNA, 20 bp, artificial
  primer
agtatagcagaatggagcaa

SEQ ID NO: 8 - DNA, 21 bp, artificial
  primer
ggaaggcaatgtggtgagggt

SEQ ID NO: 9 - DNA, 21 bp, artificial
  primer
ctgccgaaggaggagagagtg

SEQ ID NO: 10 - DNA, 21 bp, artificial
  primer
gtttccatcagctttgtagta

SEQ ID NO: 11 - DNA, 21 bp, artificial
  primer
aggaacatctactccgcagac

SEQ ID NO: 12 - DNA, 20 bp, artificial
  primer
tgaaaagatcctcctgggtg

SEQ ID NO: 13 - DNA, 23 bp, artificial
  MuSK antisense oligodeoxynucleotide
gaatgttgacgagctctctcatg

SEQ ID NO: 14 - DNA, 23 bp, artificial
  MuSK scrambled antisense oligodeoxynucleotide
tactatggatcgtctgcgcatag

SEQ ID NO: 15 - DNA, 19 bp, artificial
  primer
tgcgcctatgttggagcaa

SEQ ID NO: 16 - DNA, 20 bp, artificial
  primer
cctctgctctctcgcacatg

SEQ ID NO: 17 - DNA, 20 bp, artificial
  probe
tgcctgcagacagacccagc

SEQ ID NO: 18 - DNA, 2298 bp, Rattus norvegicus
atgagagagctcgtcaacattccactgttacagatgctcaccctggttgccttcagcgggaccgagaaacttccaaaagcccctgtcatcaccacgcctcttgaaactgtagatgccttagttgaagaagtggcgactttcatgtgcgccgtggaatcctaccctcagcctgaaatttcttggaccagaaataaaattctcatcaagctgtttgacacccgctacagcatccgagagaacggtcagctcctcaccatcctgagtgtggaggacagtgatgatggcatctactgctgcacagccaacaatggagtgggaggagcggtggaaagttgtggcgccctgcaagtgaagatgaagcctaaaataactcgtcctcccatcaatgtaaaaataattgagggattgaaagcagtcctaccgtgcactacgatgggtaaccccaagccatccgtgtcctggattaagggggacagtgctctcagggaaaattccaggattgcagttcttgaatctgggagtttaaggatccataatgtgcaaaaggaagacgcaggacagtaccgatgtgtggcaaaaaacagcctgggcacagcttactccaaactggtgaagctggaagtggaggaatggagcaaatcacagaaagaaagcaaaggctactgtgcccagtacagaggggaggtgtgtgatgccgtcctggtgaaagactctcttgtcttcttcaacacctcctatcccgaccctgaggaggcccaagagctgctgatccacactgcgtggaatgaactcaaggctgtgagcccactctgccgaccagctgccgaggctctgctgtgtaatcacctcttccaggagtgcagccctggagtgctacctactcctatgcccatttgcagagagtactgcttggcagtaaaggagctcttctgtgcaaaggaatggctggcaatggaagggaagacccaccgcggactctacagatccgggatgcatttcctcccggtcccggagtgcagcaagcttcccagcatgcaccaggaccccacagcctgcacaagactgccgtatttagcattcccgtccataacgtcctccaagccgagcgtggacattccaaacctgcctgcctccacgtcttccttcgccgtctcgcctgcgtactccatgactgtcatcatctccatcatgtcctgctttgcggtgtttgctctcctcaccatcactactctctattgctgccgaaggaggagagagtggaaaaataagaaaagagagtcggcagcggtgaccctcaccacattgccttccgagctcctgctggacaggctgcatcccaaccccatgtaccagaggatgccactccttctgaatcccaagttgctcagcctggagtatccgaggaataacatcgagtatgtcagagacatcggagagggagcgtttggaagggtctttcaagcgagggccccaggcttgcttccttatgaacccttcactatggtggctgtgaagatgctgaaggaggaggcctccgcagatatgcaggcagactttcagagggaggcagccctcatggcggagtttgacaaccccaacattgtgaagctcttaggtgtgtgtgctgttgggaagccaatgtgcctgctctttgaatatatggcctatggtgacctcaatgagttcctccgaagcatgtcccctcacactgtgtgcagcctcagccacagtgacctgtccacgagggctcgggtgtccagccctggtcctccacccctgtcttgtgcggaacagctctgtattgccaggcaagtggcagctggcatggcctacctgtcggagcgcaagtttgtccatcgggacttagctaccaggaactgcctggttggagagaacatggtggtgaaaattgcagactttggcctctctaggaacatctactccgcagactactacaaagctgatggaaacgatgctatacctatccgctggatgccacccgagtctatcttctacaaccgctacaccacggagtcagatgtgtgggcttatggcgtggtcctctgggagatcttctcctatggactgcagccctactatggaatggcccatgaggaggtcatttactatgtgagagatggtaacatccttgcctgccctgagaactgtcccttggaactgtacaaccttatgcgcctatgttggagcaagctgcctgcagacagacccagcttctgcagtatccaccggatcctgcagcgcatgtgcgagagagcagagggaacggtaggcgtctaa

SEQ ID NO: 19 - protein, 765 aa, Rattus norvegicus
MRELVNIPLLQMLTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSALRENSRIAVLESGSLRIHNVQKEDAGQYRCVAKNSLGTAYSKLVKLEVEEWSKSQKESKGYCAQYRGEVCDAVLVKDSLVFFNTSYPDPEEAQELLIHTAWNELKAVSPLCRPAAEALLCNHLFQECSPGVLPTPMPICREYCLAVKELFCAKEWLAMEGKTHRGLYRSGMHFLPVPECSKLPSMHQDPTACTRLPYLAFPSITSSKPSVDIPNLPASTSSFAVSPAYSMTVIISIMSCFAVFALLTITTLYCCRRRREWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSTRARVSSPGPPPLSCAEQLCIARQVAAGMAYLSERKFVHRDLATRNCLVGENMVVKIADFGLSRNIYSADYYKADGNDAIPIRWMPPESIFYNRYTTESDVWAYGVVLWEIFSYGLQPYYGMAHEEVIYYVRDGNILACPENCPLELYNLMRLCWSKLPADRPSFCSIHRILQRMCERAEGTVGV

SEQ ID NO: 20 - DNA, 2583 bp, Rattus norvegicus
atgagagagctcgtcaacattccactgttacagatgctcaccctggttgccttcagcgggaccgagaaacttccaaaagcccctgtcatcaccacgcctcttgaaactgtagatgccttagttgaagaagtggcgactttcatgtgcgccgtggaatcctaccctcagcctgaaatttcttggaccagaaataaaattctcatcaagctgtttgacacccgctacagcatccgagagaacggtcagctcctcaccatcctgagtgtggaggacagtgatgatggcatctactgctgcacagccaacaatggagtgggaggagcggtggaaagttgtggcgccctgcaagtgaagatgaagcctaaaataactcgtcctcccatcaatgtaaaaataattgagggattgaaagcagtcctaccgtgcactacgatgggtaaccccaagccatccgtgtcctggattaagggggacagtgctctcagggaaaattccaggattgcagttcttgaatctgggagtttaaggatccataatgtgcaaaaggaagacgcaggacagtaccgatgtgtggcaaaaaacagcctgggcacagcttactccaaactggtgaagctggaagtggaggtttttgcaagaatcctgcgtgctcctgaatcccacaatgtcacctttggttcctttgtaaccctacgctgcacagcaataggcatgcctgtccccaccatcagctggattgaaaacggaaatgctgtttcttcaggttccattcaagagaatgtgaaagaccgagtgattgactcaagactccagctctttatcacaaagccaggactctacacatgcatagctaccaataagcatggagagaaattcagtaccgcaaaggctgcagccactgtcagtatagcagaatggagcaaatcacagaaagaaagcaaaggctactgtgcccagtacagaggggaggtgtgtgatgccgtcctggtgaaagactctcttgtcttcttcaacacctcctatcccgaccctgaggaggcccaagagctgctgatccacactgcgtggaatgaactcaaggctgtgagcccactctgccgaccagctgccgaggctctgctgtgtaatcacctcttccaggagtgcagccctggagtgctacctactcctatgcccatttgcagagagtactgcttggcagtaaaggagctcttctgtgcaaaggaatggctggcaatggaagggaagacccaccgcggactctacagatccgggatgcatttcctcccggtcccggagtgcagcaagcttcccagcatgcaccaggaccccacagcctgcacaagactgccgtatttagcattcccgtccataacgtcctccaagccgagcgtggacattccaaacctgcctgcctccacgtcttccttcgccgtctcgcctgcgtactccatgactgtcatcatctccatcatgtcctgctttgcggtgtttgctctcctcaccatcactactctctattgctgccgaaggaggagagagtggaaaaataagaaaagagagtcggcagcggtgaccctcaccacattgccttccgagctcctgctggacaggctgcatcccaaccccatgtaccagaggatgccactccttctgaatcccaagttgctcagcctggagtatccgaggaataacatcgagtatgtcagagacatcggagagggagcgtttggaagggtctttcaagcgagggccccaggcttgcttccttatgaacccttcactatggtggctgtgaagatgctgaaggaggaggcctccgcagatatgcaggcagactttcagagggaggcagccctcatggcggagtttgacaaccccaacattgtgaagctcttaggtgtgtgtgctgttgggaagccaatgtgcctgctctttgaatatatggcctatggtgacctcaatgagttcctccgaagcatgtcccctcacactgtgtgcagcctcagccacagtgacctgtccacgagggctcgggtgtccagccctggtcctccacccctgtcttgtgcggaacagctctgtattgccaggcaagtggcagctggcatggcctacctgtcggagcgcaagtttgtccatcgggacttagctaccaggaactgcctggttggagagaacatggtggtgaaaattgcagactttggcctctctaggaacatctactccgcagactactacaaagctgatggaaacgatgctatacctatccgctggatgccacccgagtctatcttctacaaccgctacaccacggagtcagatgtgtgggcttatggcgtggtcctctgggagatcttctcctatggactgcagccctactatggaatggcccatgaggaggtcatttactatgtgagagatggtaacatccttgcctgccctgagaactgtcccttggaactgtacaaccttatgcgcctatgttggagcaagctgcctgcagacagacccagcttctgcagtatccaccggatcctgcagcgcatgtgcgagagagcagagggaacggtaggcgtctaa

SEQ ID NO: 21 - DNA, 20 bp, artificial
  primer
atgagagagctcgtcaacat

SEQ ID NO: 22 - DNA, 20 bp, artificial
  primer
tgaaaagatcctcctgggtg